(19) United States
(12) Patent Application Publication
(10) Pub. No.: US 2017/0105608 A1
(43) Pub. Date: Apr. 20, 2017

(54) ENDOSCOPE SYSTEM

(71) Applicant: OLYMPUS CORPORATION, Tokyo (JP)

(72) Inventors: Yasuhito KURA, Tokyo (JP); Hideyuki KUGIMIYA, Tokyo (JP); Takeo SUZUKI, Tokyo (JP); Kento HASHIMOTO, Tokyo (JP)

(73) Assignee: OLYMPUS CORPORATION, Tokyo (JP)

(21) Appl. No.: 15/391,185

(22) Filed: Dec. 27, 2016

Related U.S. Application Data
(63) Continuation of application No. PCT/JP2015/067700, filed on Jun. 19, 2015.

(30) Foreign Application Priority Data
Jun. 27, 2014 (JP) 2014-133139

(51) Int. Cl. A61B 1/00; H04N 5/225; G02B 23/24; A61B 1/05; A61B 1/018

(52) U.S. Cl. CPC A61B 1/00181; A61B 1/05; A61B 1/00009; A61B 1/018; A61B 1/0005; G02B 23/2484; H04N 5/2256; H04N 2005/2255

(57) ABSTRACT

An endoscope system includes an insertion portion, a support portion, a first subject image acquisition portion, a second subject image acquisition portion, a third subject image acquisition portion, an image generation section, and an image processing section. The image processing section can switch between a first mode of arranging lateral images around a forward image in a continuously connected state and a second mode of arranging the lateral images and the forward image in a separated. The image processing section continuously connects a third image in a non-display region to the forward image in the first mode, and arranges the lateral images side by side with both sides of the forward image and arranges the third image adjacent to the forward image in the second mode.

### CROSS REFERENCE TO RELATED APPLICATION

This application is a continuation application of PCT/JP2015/067700 filed on Jun. 19, 2015 and claims benefit of Japanese Application No. 2014-133139 filed in Japan on Jun. 27, 2014, the entire contents of which are incorporated herein by this reference.

### BACKGROUND OF THE INVENTION

1. Field of the Invention

The present invention relates to an endoscope system capable of observing a forward field of view and a lateral field of view at the same time.

2. Background Art

Conventionally, endoscopes including insertion portions formed in elongated shapes are widely used in, for example, a medical field and an industrial field. Among these, in a medical endoscope used in the medical field, an elongated insertion portion can be inserted into a body cavity as a subject to observe an organ in the body cavity, and a treatment instrument inserted into a treatment instrument insertion channel included in the endoscope can be used as necessary to conduct various treatments. In an industrial endoscope used in the industrial field, an elongated insertion portion can be inserted into an object, such as a jet engine and a factory pipe, to observe and inspect a state in the object, such as scratches and corrosion.

In recent years, various proposals have been disclosed regarding constitutive schemes for further facilitating the inspection and the like performed by using the conventional endoscopes. For example, Japanese Patent No. 4782900 and the like propose various endoscopes capable of acquiring a forward field of view image in which a forward direction of an insertion direction (insertion axis direction) of an endoscope insertion portion is an observation field of view and also capable of acquiring at the same time a lateral field of view image in which a lateral direction of the endoscope insertion portion is an observation field of view.

In an endoscope system disclosed in Japanese Patent No. 4782900 and the like, one image pickup device can acquire, at the same time, the forward field of view image in which the forward direction of the insertion direction (insertion axis direction) of the endoscope insertion portion is the observation field of view and the lateral field of view image in which the lateral direction of the endoscope insertion portion is the observation field of view, and both of the acquired images can be displayed in an annular shape in one screen to display an endoscopic image with a wide field of view. Furthermore, an endoscope system is also proposed, wherein field of view images in a plurality of directions are acquired based on the configuration as described above, and a wide field of view image can be displayed by displaying a plurality of images side by side.

### SUMMARY OF THE INVENTION

An aspect of the present invention provides an endoscope system including: an insertion portion inserted into a subject; a support portion protruding forward from a distal end surface of the insertion portion; a first subject image acquisition portion provided on the insertion portion and configured to acquire a first subject image from a forward region including a forward direction of the insertion portion; a second subject image acquisition portion provided on the insertion portion and configured to acquire a second subject image from a lateral region including a radial direction of the insertion portion, the lateral region being at least partially different from the forward region; a third subject image acquisition portion provided on the insertion portion and configured to acquire a third subject image from a third region that is a region in the forward region shielded by the support portion; an image generation section configured to generate a forward image based on the first subject image, lateral images based on the second subject image, and a third image based on the third subject image; and an image processing section configured to execute a process of arranging the lateral images around the forward image and arranging the third image in a non-display region of the lateral images generated by being shielded by the support portion, wherein the image processing section can switch between a first mode of arranging the lateral images around the forward image in a state in which parts of the lateral images including both sides of the forward image are continuously connected and a second mode of arranging the lateral images side by side with the forward image in a state in which the parts including both sides of the forward image are separated, and the image processing section executes a process of arranging the third image in the non-display region in a state in which the third image is continuously connected to the forward image in the first mode and a process of arranging the lateral images side by side with both sides of the forward image and arranging the third image adjacent to a region different from both sides of the forward image in the second mode.

### BRIEF DESCRIPTION OF THE DRAWINGS

FIG. 1 is a schematic configuration of an overall configuration of an endoscope system according to a first embodiment of the present invention;

FIG. 2 is an enlarged vertical cross-sectional view of main parts showing a schematic configuration of the entire endoscope system of FIG. 1 and showing a cross section of an internal configuration of a distal end portion of an insertion portion of an endoscope in the endoscope system;

FIG. 3 is an enlarged perspective view of main parts showing an appearance of the distal end portion of the insertion portion of the endoscope in the endoscope system of FIG. 1;

FIG. 4 is a diagram showing an example of display of an endoscopic image in a first display form that can be displayed by a display apparatus in the endoscope system of FIG. 1;

FIG. 5 is a diagram showing an example of display of an endoscopic image in a second display form that can be displayed by the display apparatus in the endoscope system of FIG. 1;

FIG. 6 is a diagram showing a modification of the endoscopic image in the first display form that can be displayed by the display apparatus in the endoscope system of FIG. 1;

FIG. 7 is a diagram showing a modification of the endoscopic image in the second display form that can be displayed by the display apparatus in the endoscope system of FIG. 1;

FIG. 8 is a diagram showing an example of display when expansion operation or contraction operation of the image is performed in a display screen displaying the image in the second display form of FIG. 5 by the display apparatus in the endoscope system of FIG. 1;

FIG. 9 is a diagram showing an example of display when movement operation of an image region is performed in the display screen displaying the image in the second display form of FIG. 5 by the display apparatus in the endoscope system of FIG. 1;

FIG. 10 is a diagram showing an example of display when rotation operation of the image is performed in the display screen displaying the image in the second display form of FIG. 5 by the display apparatus in the endoscope system of FIG. 1;

FIG. 11 is a diagram showing an example of display when movement operation of the image (entire image) is performed in the display screen displaying the image in the second display form of FIG. 5 by the display apparatus in the endoscope system of FIG. 1;

FIG. 12 is an external perspective view showing a first modification of the distal end portion of the endoscope insertion portion in the endoscope system according to the first embodiment of the present invention;

FIG. 13 is an external perspective view showing a second modification of the distal end portion of the endoscope insertion portion in the endoscope system according to the first embodiment of the present invention;

FIG. 14 is a diagram showing an example of the first display form of the endoscopic image displayed on the display screen of the display apparatus in the endoscope system including the distal end portion of the endoscope insertion portion of the first modification of FIG. 12;

FIG. 15 is a diagram showing an example of the second display form of the endoscopic image displayed on the display screen of the display apparatus in the endoscope system including the distal end portion of the endoscope insertion portion of the first modification of FIG. 12;

FIG. 16 is an external perspective view showing another modification of the distal end portion of the endoscope insertion portion in the endoscope system according to the first embodiment of the present invention;

FIG. 17 is a diagram showing an example of the display form of the endoscopic image displayed on the display screen of the display apparatus in the endoscope system including the distal end portion of the endoscope insertion portion of another modification of FIG. 16;

FIG. 18 is a diagram showing another example of the display form of the endoscopic image displayed on the display screen of the display apparatus in the endoscope system according to the first embodiment of the present invention;

FIG. 19 is a diagram showing another different example of the display form of the endoscopic image displayed on the display screen of the display apparatus in the endoscope system according to the first embodiment of the present invention;

FIG. 20 is a diagram showing yet another different example of the display form of the endoscopic image displayed on the display screen of the display apparatus in the endoscope system according to the first embodiment of the present invention;

FIG. 21 is an enlarged vertical cross-sectional view of main parts showing a schematic configuration of an entire endoscope system according to a second embodiment of the present invention and showing an internal configuration of a distal end portion of an insertion portion of an endoscope in the endoscope system;

FIG. 22 is a conceptual diagram showing the distal end portion of the insertion portion of the endoscope in the endoscope system of FIG. 21;

FIG. 23 is a diagram showing an example of display of the endoscopic image that can be displayed by the display apparatus in the endoscope system of FIG. 21;

FIG. 24 is a diagram showing another example of display of the endoscopic image that can be displayed by the display apparatus in the endoscope system of FIG. 21; and

FIG. 25 is a diagram showing a list of the examples of display form of the endoscopic image displayed on the display apparatus (display section) in the endoscope system of FIG. 21.

### DETAILED DESCRIPTION OF THE PREFERRED EMBODIMENT(S)

Hereinafter, the present invention will be described based on illustrated embodiments. Scaling of each constituent element may vary in each drawing used in the following description in order to illustrate each constituent element in a size that allows recognizing the constituent element on the drawing. Therefore, quantities of the constituent elements described in the drawings, shapes of the constituent elements, ratios of sizes of the constituent elements, and relative positional relationships between respective constituent elements of the present invention are not limited only to the illustrated modes.

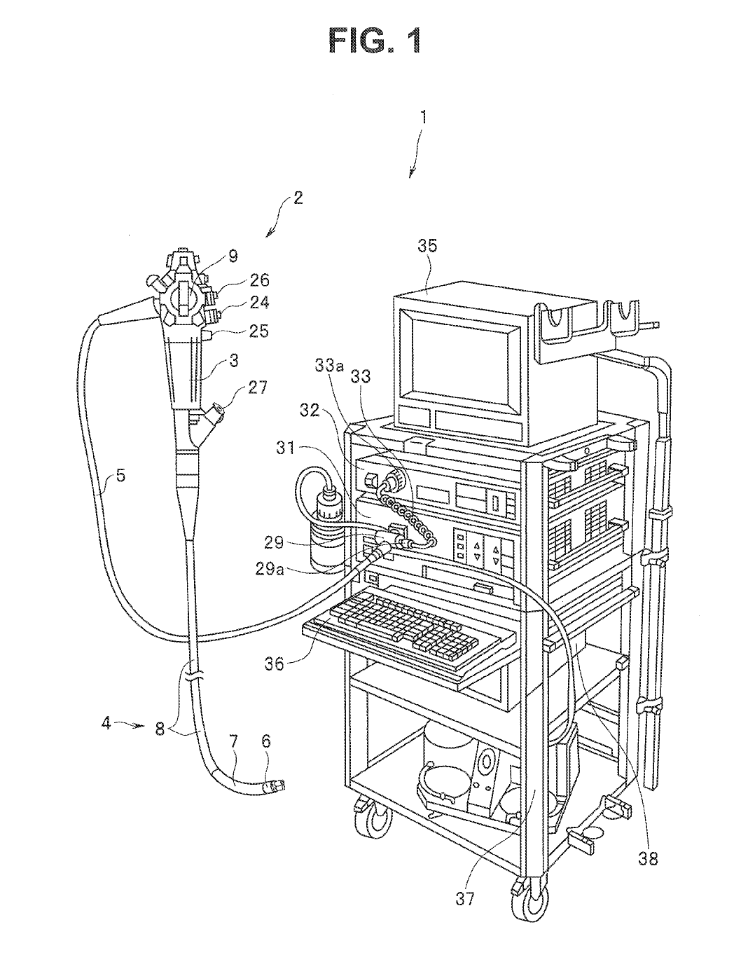

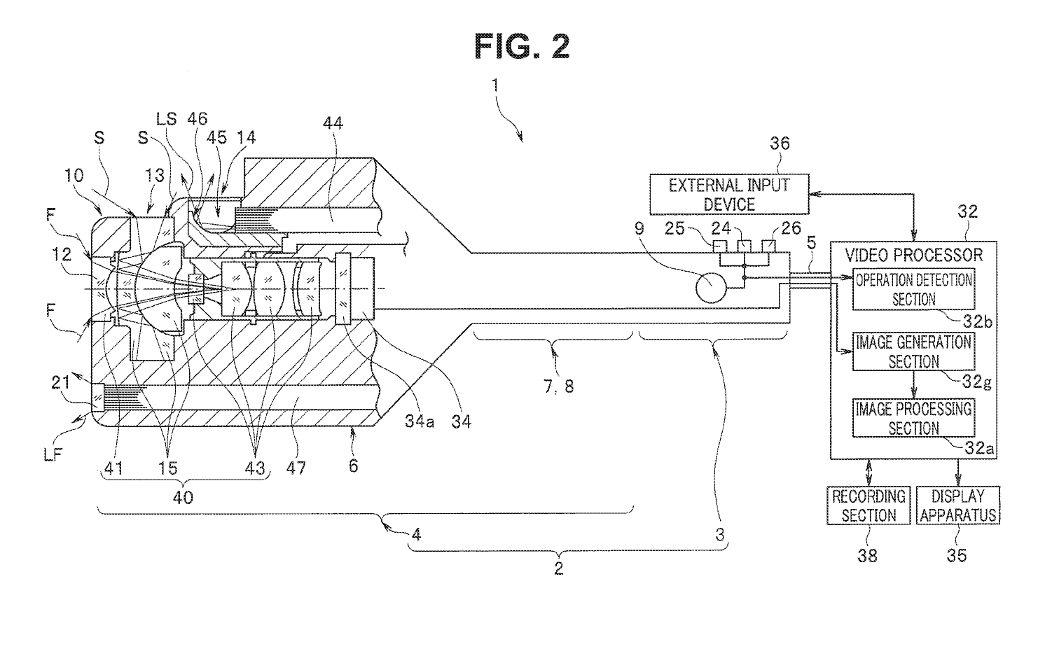

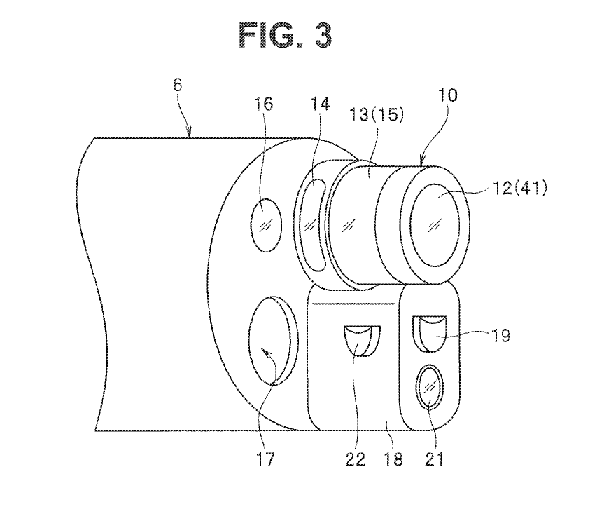

FIGS. 1 and 2 are diagrams showing an endoscope system according to a first embodiment of the present invention. Among these, FIG. 1 is a schematic configuration diagram of an overall configuration of the endoscope system of the present embodiment. FIG. 2 is an enlarged vertical cross-sectional view of main parts showing a schematic configuration of the entire endoscope system of FIG. 1 and showing a cross section of an internal configuration of a distal end portion of an insertion portion of an endoscope in the endoscope system. FIG. 3 is an enlarged perspective view of main parts showing an appearance of the distal end portion of the insertion portion of the endoscope in the endoscope system of FIG. 1.

First, the overall configuration of the endoscope system of the first embodiment of the present invention will be simply described by mainly using FIGS. 1 and 2.

An endoscope system 1 of the present embodiment includes an endoscope 2, a light source apparatus 31, a video processor 32, a display apparatus 35, a keyboard 36 that is an external input device, a stand 37, and the like.

The endoscope 2 includes an operation portion 3, an insertion portion 4, a universal cord 5, and the like. Among these, the insertion portion 4 is an elongated tubular constituent unit formed by consecutively connecting a distal end portion 6, a bending portion 7, and a flexible tube portion 8 in the order from a distal end. A proximal end of the insertion portion 4 is consecutively connected to a distal end of the operation portion 3. The insertion portion 4 is a constituent portion inserted into a lumen, that is, a body cavity, of a subject during the use of the endoscope 2.

The flexible tube portion 8 of the insertion portion 4 is formed by using a long tubular member that is flexible and hollow. A proximal end side is consecutively connected to the distal end of the operation portion 3, and a distal end side is consecutively connected to a proximal end of the bending portion 7. Various signal lines, a light guide cable, a treatment instrument channel, and the like extended from the distal end portion 6 are inserted and arranged inside of the flexible tube portion 8.

The bending portion 7 is a constituent portion formed to be bendable in an up and down direction and a left and right direction relative to an insertion axis of the insertion portion 4. The bending portion 7 is configured by continuously connecting, for example, a plurality of bending pieces just like the configuration conventionally applied in a general endoscope. Therefore, a detailed configuration and an internal configuration of the bending portion are not illustrated. A proximal end side of the bending portion 7 is consecutively connected to a distal end of the flexible tube portion 8, and a distal end side is consecutively connected to a proximal end of the distal end portion 6. Note that the bending portion 7 can be bent, for example, in the up and down direction and the left and right direction by operating a bending operation knob 9 of the operation portion 3 described later.

The distal end portion 6 is located on a most distal end side of the insertion portion 4 and is configured by a rigid member. The distal end portion 6 is a constituent unit in which various constituent members are arranged on a distal end portion and inside. Note that a detailed configuration of the distal end portion 6 will be described later (see FIGS. 2 and 3).

The operation portion 3 is grasped by a hand of a user during the use and is a constituent portion configured to support the endoscope 2. A plurality of operation members for performing various operations are located on one of end portion peripheral surfaces of the operation portion 3. The plurality of operation members are respectively located on parts in a range that the fingers can reach when the user grasps the operation portion 3. Specific examples of the plurality of operation members include an air/liquid feeding operation button 24, a scope switch 25, a suction operation button 26, and the bending operation knob 9.

The air/liquid feeding operation button 24 is an operation member for selectively ejecting air, liquid, and the like for cleaning, from a forward field of view observation window nozzle portion 19 and lateral field of view observation window nozzle portions 22 (described later; see FIG. 3) provided on the distal end portion 6 of the insertion portion 4.

The suction operation button 26 is an operation member for performing suction operation to recover mucus or the like in the body cavity from a channel distal end opening portion 17 (described later; see FIG. 3) provided on the distal end portion 6 of the insertion portion 4.

The scope switch 25 is an operation member for switching a display form when a display apparatus 35 that is a display section is used to display an endoscopic image acquired by an image pickup section (including an objective optical system 40, an image pickup device 34, and the like; details will be described later; see FIG. 2) provided on the distal end portion 6 of the insertion portion 4. Note that details of the display form of the endoscopic image displayed by using the display apparatus 35 in the endoscope system 1 of the present embodiment will be described later.

The respective operation members, such as the air/liquid feeding operation button 24, the scope switch 25, and the suction operation button 26, are linked with a plurality of respectively corresponding operation switches inside of the operation portion 3. The plurality of operation switches are mounted on an internal main substrate (not shown) of the operation portion 3. The internal main substrate of the operation portion 3 is electrically connected with the video processor 32. According to the configuration, when the respective operation members are operated by the user, predetermined corresponding instruction signals are generated from the operation switches linked with the respective operation members. The instruction signals are transmitted to the video processor 32, and corresponding control processes are executed.

On the other hand, the bending operation knob 9 is a rotation operation member for operating a bending operation mechanism (not shown) located inside of the operation portion 3.

Although the operation members provided on the operation portion 3 include various operation members other than the operation members illustrated above, the individual operation members are the same as the operation members applied in a conventional general endoscope, and the detailed description and the illustration are omitted.

A treatment instrument insertion port 27 is provided on a part closer to the distal end of the operation portion 3, the treatment instrument insertion port 27 protruding outward from a lateral direction. The treatment instrument insertion port 27 communicates with the treatment instrument channel (not shown) inserted and arranged inside of the operation portion 3 and the insertion portion 4. The treatment instrument channel is formed by a tubular member, such as a tube, inserted and arranged inside of the operation portion 3 and inside of the insertion portion 4 and reaching the channel distal end opening portion 17 opening on a front surface of the distal end portion 6 of the insertion portion 4.

When the user performs a treatment or the like using a treatment instrument not shown through the endoscope 2 of the endoscope system 1, the user inserts a predetermined treatment instrument from the treatment instrument insertion port 27 and inserts the treatment instrument into the treatment instrument channel. The user then causes a distal end part of the treatment instrument to protrude forward in an insertion direction from the channel distal end opening portion 17. In this way, the distal end part of the treatment instrument can reach a desired part to be inspected in the body cavity, and therefore, various treatments, such as therapy, can be conducted.

The universal cord 5 extends outward from a side portion of the operation portion 3. The universal cord 5 is a cable member in which a plurality of signal lines, a light guide cable, an air/liquid feeding tube, a suction tube, and the like are inserted and arranged inside. A connector 29 is provided on a distal end of the universal cord 5.

A fluid conduit connection pipe sleeve (not shown), a light guide pipe sleeve (not shown) that is an illumination light supply end portion, an electrical contact portion 29a, and the like are provided on the connector 29. Here, an air/liquid feeding apparatus (not shown) is detachably connected to the fluid conduit connection pipe sleeve. The light source apparatus 31 is detachably connected to the light guide pipe sleeve. One end of a connection cable 33 is detachably connected to the electrical contact portion 29a.

A connector 33a is provided on the other end of the connection cable 33, and the connector 33a is connected to the video processor 32 that is signal processing and control means.

The light source apparatus 31 is a constituent unit configured to generate illumination light. The light guide cable is connected to the light source apparatus 31 as described above. The light guide cable is inserted inside of the universal cord 5 and then inserted inside of the operation portion 3 and the insertion portion 4. The light guide cable reaches the inside of the distal end portion 6 of the insertion portion 4. In this way, illumination light emitted from the light source apparatus 31 is guided by the light guide cable to the distal end portion 6.

Here, although not shown, a distal end side of the light guide cable is branched at a predetermined part inside of the insertion portion 4, for example. Respective distal ends of one of the cables as a lateral field of view illumination light guide 44 and the other cable as a forward field of view illumination light guide 47 are located at respective predetermined parts inside of the distal end portion 6.

More specifically, the distal end of the lateral field of view illumination light guide 44 is arranged near lateral field of view illumination windows 14 of the distal end portion 6. In this way, the illumination light guided from the light source apparatus 31 to the lateral field of view illumination light guide 44 is emitted outward from the lateral field of view illumination windows 14 to illuminate a lateral field of view (see FIGS. 2 and 3).

The distal end of the forward field of view illumination light guide 47 is connected to forward field of view illumination windows (16, 21) provided on a distal end surface of the distal end portion 6. In this way, the illumination light guided from the light source apparatus 31 to the forward field of view illumination light guide 47 is emitted outward from the forward field of view illumination windows (16, 21) to illuminate a forward field of view (see FIGS. 2 and 3).

The video processor 32 is control means for comprehensively controlling the present endoscope system 1 and is signal processing means for processing various electrical signals.

The video processor 32 supplies, for example, control signals for driving the image pickup section and the like (described later; see FIG. 2) and receives instruction signals from various operation members of the operation portion 3 to output corresponding control signals. The video processor 32 receives, for example, an image signal outputted from the image pickup section (image pickup device 34) and executes predetermined signal processing to generate an image signal for display and to generate image data for recording. Therefore, a plurality of substrate units configuring electronic control circuits and the like, such as an image processing section 32a configured to receive various instruction signals and apply image signal processing corresponding to various instructions to the output signals (image signals) from the image pickup section and an operation detection section 32b configured to detect instruction signals from the operation portion 3, are located inside of the video processor 32.

The display apparatus 35 (display section) is a constituent unit configured to receive the display image signal generated by the video processor 32 to continuously display endoscopic images on a display screen. Examples of the display apparatus 35 include a liquid crystal display (LCD) device, an organic electro-luminescence (OEL) display device, and other general display devices using CRT (cathode ray tube) and the like.

The keyboard 36 is electrically connected to the video processor 32 and is an external input device for inputting an instruction to the video processor 32 and for inputting various information such as patient information. Note that other than the keyboard 36, examples of the external input device connected to the video processor 32 include a pointing device, such as a mouse, a trackball, a joystick, and a touch pad, a foot switch, a voice input apparatus, and a touch panel located on the display screen of the display apparatus 35, and various existing devices can be appropriately applied. Not only one external input device, but also a plurality of external input devices may be provided at the same time.

The stand 37 is a housing and mounting apparatus for mounting constituent units, such as the light source apparatus 31, the video processor 32, the display apparatus 35, and the external input device (keyboard 36), and for temporarily mounting the endoscope 2 not in use by suspending the endoscope 2.

This is the schematic configuration of the endoscope system 1 of the present embodiment. Configurations not described above are the same as configurations in a conventionally and generally implemented endoscope system.

Next, the detailed configuration of the distal end portion 6 will be described by mainly using FIGS. 2 and 3. As shown in FIGS. 2 and 3, a cylindrical portion 10 is formed on the distal end portion 6 of the insertion portion 4 of the endoscope 2, the cylindrical portion 10 protruding forward from an upper part of the distal end surface and formed in a substantially cylindrical shape. A support portion 18 is formed on an adjacent part of a lower side of the cylindrical portion 10, the support portion 18 protruding forward from the distal end surface of the distal end portion 6 just like the cylindrical portion 10. The support portion 18 is a support member for supporting the cylindrical portion 10 and has a function of shielding an unnecessary part of a field of view range by limiting a lateral field of view range to prevent some structures and the like of the distal end portion 6 from being displayed as an endoscopic image.

A forward field of view observation window 12 configuring part of a first optical system, a lateral field of view observation window 13 configuring part of a second optical system, and the lateral field of view illumination windows 14 are formed on the cylindrical portion 10.

The forward field of view observation window 12 is an opening window formed on a front surface of the cylindrical portion 10 to observe the forward field of view. A first lens 41 of the objective optical system 40 is fixed and arranged on the forward field of view observation window 12. The forward field of view observation window 12 serves as an opening for receiving a light flux entered from the front in the insertion direction of the endoscope 2 and guiding the light flux to the objective optical system 40. Here, arrows F illustrated in FIG. 2 show an incident light flux from the front.

Here, a direction facing the forward field of view will be called a first direction. The forward field of view observation window 12 functions as a first subject image acquisition portion configured to acquire a first subject image (called a forward subject image or a first subject image) that is a forward field of view image from a forward region including the forward direction of the insertion portion that is the first direction. That is, the first subject image is a field of view image of a first region including the forward direction substantially parallel to a longitudinal direction of the insertion portion 4.

The first subject image acquisition portion is a forward subject image acquisition portion configured to acquire a field of view image of a region including the forward direction of the insertion portion 4. Note that the forward field of view observation window 12 that is the first subject image acquisition portion is provided on the insertion portion 4 (on the cylindrical portion 10 of the distal end portion 6 of the insertion portion 4). That is, the first subject image acquisition portion is arranged in a direction in which the insertion portion 4 is inserted into the longitudinal direction distal end portion of the distal end portion 6 of the insertion portion 4.

The lateral field of view observation window 13 is an annular opening window formed substantially throughout the whole circumference along a peripheral surface of a part in the middle of the cylindrical portion 10 to observe the lateral field of view. A reflective optical system 15, details of which will be described later, configuring part of the objective optical system 40 is fixed and arranged in an internal space of the cylindrical portion 10 facing the lateral field of view observation window 13. The lateral field of view observation window 13 serves as an opening for receiving a light flux entering from the lateral direction of the endoscope 2 and guiding the light flux to the objective optical system 40. Here, arrows S illustrated in FIG. 2 show an incident light flux from the lateral direction.

Here, a direction facing the lateral field of view will be called a second direction different from the first direction (the direction facing the forward field of view). The lateral field of view observation window 13 functions as a second subject image acquisition portion configured to acquire a second subject image (called a lateral subject image or a second subject image) that is a lateral field of view image from a lateral region including the lateral direction of the insertion portion 4 that is the second direction. That is, the second subject image is a field of view image of a second region including the lateral direction of the insertion portion 4 that is a radial direction of the insertion portion 4, that is, a direction inclined relative to the longitudinal direction of the insertion portion 4 (for example, a direction substantially perpendicular to the longitudinal direction of the insertion portion 4). Note that in the present embodiment, a region of part of the lateral field of view image, more specifically, a field of view closer to the lower side (lower field of view) in the lateral direction, is in a non-display state as described later, and the second direction does not include the lower field of view image.

Note that the lateral region (second region) is a region at least partially different from the forward region (first region), and part of the lateral region (second region) may or may not overlap with the forward region (first region).

The second subject image acquisition portion is a lateral subject image acquisition portion configured to acquire a field of view image of a region including the lateral direction of the insertion portion 4. Note that the lateral field of view observation window 13 that is the second subject image acquisition portion is provided on the insertion portion 4 (on the cylindrical portion 10 of the distal end portion 6 of the insertion portion 4). That is, the second subject image acquisition portion is arranged to surround a circumferential direction of the distal end portion 6 of the insertion portion 4. The second subject image acquisition portion (lateral field of view observation window 13) is arranged closer to the proximal end than the first subject image acquisition portion (forward field of view observation window 12) on the distal end portion 6 of the insertion portion 4.

The lateral field of view illumination windows 14 are illumination openings for receiving emission light from the light guide 44 to illuminate the lateral field of view (lateral direction of the insertion portion 4). Therefore, at least one or a plurality of lateral field of view illumination windows 14 are provided on parts adjacent to the lateral field of view observation window 13, near a proximal end of the cylindrical portion 10. The present embodiment illustrates an example in which two lateral field of view illumination windows 14 are provided on a circumferential surface of the cylindrical portion 10, at an interval of 180 degrees around a center axis. Note that only one of the lateral field of view illumination windows 14 is illustrated in FIG. 3, and the other is provided on a position not illustrated.

The lateral field of view illumination windows 14 open in a circumferential direction of the cylindrical portion 10 in the radial direction of the insertion portion 4, that is, in the lateral direction of the insertion portion 4 that is a direction inclined relative to an axis direction of the insertion portion 4. In this case, illumination light emitted from the lateral field of view illumination windows 14 is not emitted toward the side where the support portion 18 is located. Therefore, the illumination light from the lateral field of view illumination windows 14 is emitted toward a region excluding a lower side provided with the support portion 18 in the circumferential direction of the cylindrical portion 10. Here, an arrow LS illustrated in FIG. 2 shows the illumination light emitted in the lateral direction from the lateral field of view illumination windows 14.

As shown in FIG. 2, the image pickup section including the objective optical system 40, the image pickup device 34, and the like and the distal end of the lateral field of view illumination light guide 44 are located inside of the cylindrical portion 10.

The objective optical system 40 configuring part of the image pickup section is an image formation optical system including a plurality of optical lenses. In the objective optical system 40, the first lens 41, the reflective optical system 15, and rear group lenses 43 are sequentially arranged from a distal end side of the cylindrical portion 10 such that respective lens optical axes coincide, and respective lenses are arranged in rotational symmetry. Here, the optical axis of the objective optical system 40 is set to substantially coincide with the center axis of the cylindrical portion 10. Note that the respective lenses configuring the objective optical system 40 are fixed and held at fixing parts, such as fixing and holding portions and lens holding frames, inside of the cylindrical portion 10.

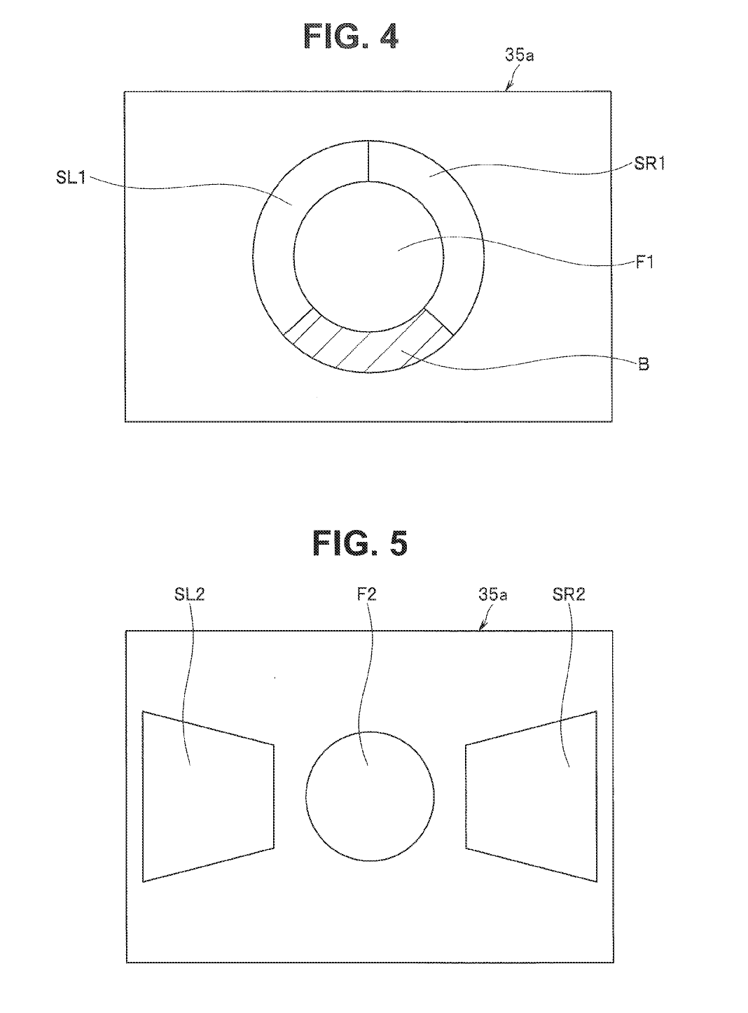

As described, the first lens 41 is fixed to the forward field of view observation window 12 that is the front surface opening window of the cylindrical portion 10. As a result, the first lens 41 is an optical system configured to observe the forward field of view of the distal end portion 6 of the insertion portion 4 that is the insertion direction of the insertion portion 4. The first lens 41 has an optical performance with a relatively wide angle of view. Note that the forward field of view observation window 12 is formed in, for example, a substantially circular shape, and the first lens 41 is also formed in a substantially circular shape. As a result, the optical image of the forward field of view (forward field of view image; first field of view image) generated by the objective optical system 40 including the first lens 41 is formed in a substantially circular shape on an image formation surface (image pickup surface) (described later; see FIG. 4).

As shown in FIG. 2, the reflective optical system 15 is formed by connecting a plurality of optical lenses. The reflective optical system 15 is an optical system configured to receive a light flux entering through the lateral field of view observation window 13 from a side direction of the insertion portion 4, bend a travel direction of the light flux by two times of surface reflection, and guide the light flux in a direction of the rear group lenses 43, that is, a direction of a light receiving surface of the image pickup device 34.

As a result, the reflective optical system 15 has a lateral optical axis substantially orthogonal to a major axis direction of the insertion portion 4 and has a predetermined view angle in which the lateral optical axis is substantially the center. The reflective optical system 15 can obtain a substantially annular observation field of view in the circumferential direction of the cylindrical portion 10 according to the lateral field of view observation window 13.

Therefore, the optical image of the lateral field of view (lateral field of view image; second field of view image) generated by the objective optical system 40 including the reflective optical system 15 and the rear group lenses 43 is formed in a substantially annular shape on the image formation surface (described later; see FIG. 4).

Here, FIG. 2 shows an outline of incident paths of each of the beam F from the subject side in the forward field of view entering the objective optical system 40 from the forward field of view observation window 12 through the first lens 41 and the beam S from the object side in the lateral field of view entering the objective optical system 40 from the lateral field of view observation window 13 through the reflective optical system 15.

Note that although the objective optical system 40 including the reflective optical system 15 and the rear group lenses 43 covers the field of view of the whole circumference of the cylindrical portion 10, the support portion 18 is provided adjacent to the cylindrical portion 10 to limit the lateral field of view range as described above. Therefore, the lateral field of view image formed on the image formation surface is an image in which part of the substantially annular shape is cut out (described later; see FIG. 4).

For example, one image pickup device 34, the light receiving surface of which faces forward, configuring another part of the image pickup section is arranged behind the objective optical system 40. The light receiving surface of the image pickup device 34 is arranged to coincide with the image formation surface on which the optical image generated by the objective optical system 40 is formed. On a front surface of the image pickup device 34, a cover glass 34a made of a flat transparent member is located parallel to the light receiving surface.

On the image formation surface of the image pickup device 34, the forward field of view image generated through the objective optical system 40 including the first lens 41 is formed in a substantially circular shape at a substantially center part. On the image pickup surface of the image pickup device 34, the lateral field of view image generated through the objective optical system 40 including the reflective optical system 15 and the rear group lenses 43 is formed in a substantially annular shape on a peripheral edge portion of the circular forward field of view image (described later; see FIG. 4).

That is, the image pickup device 34 of the image pickup section is arranged to receive the forward field of view image (first field of view image) from the forward field of view observation window 12 (first subject image acquisition portion) and the lateral field of view image (second field of view image) from the lateral field of view observation window 13 (second subject image acquisition portion) on the same surface and photoelectrically convert the images. The image pickup device 34 is electrically connected to the image processing section 32a.

Note that examples of the image pickup device 34 include photoelectric conversion devices and the like such as a CCD (charge coupled device) image sensor and a CMOS (complementary metal oxide semiconductor) image sensor.

The distal end of the lateral field of view illumination light guide 44 is arranged near the lateral field of view illumination windows 14 formed near a proximal end portion inside of the cylindrical portion 10 as shown in FIG. 2. The lateral field of view illumination light guide 44 is a constituent member configured to guide the illumination light from the light source apparatus 31 to the distal end portion 6 as described above.

Therefore, a distal end surface of the lateral field of view illumination light guide 44 is an emission end surface of the illumination light. The emission end surface is formed in, for example, a circular or elliptical shape or a polygonal shape.

A groove portion 45 formed in a substantially belt shape along the peripheral surface of the cylindrical portion 10 and formed in a concave shape in the radial direction of the cylindrical portion 10 is formed on a part facing an emission end surface of the lateral field of view illumination light guide 44. A reflecting member 46 including a reflecting surface 46a that can reflect the illumination light is located inside of the groove portion 45. As shown in FIG. 2, a cross section of the reflecting surface 46a of the reflecting member 46 is formed to have a substantially hemispheric concave surface. The reflecting surface 46a is arranged on a part facing the emission end surface of the lateral field of view illumination light guide 44.

According to the configuration, the illumination light emitted forward from the emission end surface of the lateral field of view illumination light guide 44 is reflected by the reflecting surface 46a and emitted in the lateral direction of the distal end portion 6 (cylindrical portion 10). The illumination light reflected by the reflecting surface 46a in this case is diffused in a wide range and emitted outward from the lateral field of view illumination windows 14 to illuminate the lateral field of view (see FIGS. 2 and 3).

Note that the reflecting surface 46a can be formed by providing, for example, a metal thin film made of aluminum, chromium, nickel chromium, silver, or gold.

On the other hand, the forward field of view observation window nozzle portion 19, the lateral field of view observation window nozzle portions 22, and the forward field of view illumination window 21 are located on the support portion 18.

The forward field of view observation window nozzle portion 19 is a constituent portion including an ejection portion for ejecting a cleaning solution for cleaning a front side surface of the first lens 41 of the forward field of view observation window 12. The lateral field of view observation window nozzle portions 22 are constituent portions including ejection portions for ejecting a cleaning solution for cleaning an outer surface of the reflective optical system 15 of the lateral field of view observation window 13. Note that although only one lateral field of view observation window nozzle portion 22 is illustrated in FIG. 3, a lateral field of view observation window nozzle portion 22 in the same form is also provided on a part not shown on the opposite side of the lateral field of view observation window nozzle portion 22 illustrated in FIG. 3 across the support portion 18. This allows cleaning substantially the entire region of the lateral field of view observation window 13 in the annular shape formed throughout substantially the whole circumference along the peripheral surface of the part in the middle of the cylindrical portion 10.

The forward field of view illumination window 21 is an opening window for illumination configured to emit the illumination light forward. Therefore, a distal end surface of the forward field of view illumination light guide 47 is arranged opposite to and behind the forward field of view illumination window 21 as shown in FIG. 2. As a result, the illumination light guided from the light source apparatus 31 to the forward field of view illumination light guide 47 is emitted outward and forward from the forward field of view illumination window 21 to illuminate the forward field of view. Here, an arrow LF illustrated in FIG. 2 shows the illumination light emitted in the lateral direction from the lateral field of view illumination windows 14.

The forward field of view illumination window 16 and the channel distal end opening portion 17 are located in a region other than the location parts of the cylindrical portion 10 and the support portion 18 on the distal end surface of the distal end portion 6.

Like the forward field of view illumination window 21, the forward field of view illumination window 16 is an opening window for emitting the illumination light toward the subject to be observed in the forward field of view. Although not shown, the distal end surface of the forward field of view illumination light guide 47 branched from the light guide cable is also arranged opposite to and behind the forward field of view illumination window 16. According to the similar configuration, part of the illumination light guided from the light source apparatus 31 to the forward field of view illumination light guide 47 is guided to the forward field of view illumination window 16 and emitted outward and forward from here to similarly illuminate the forward field of view. The channel distal end opening portion 17 is a distal end side opening of the treatment instrument channel.

Although the endoscope system 1 of the present embodiment includes various constituent members other than the components described above, the other components have the same configurations as in a conventionally and generally implemented endoscope system, and the detailed description and the illustration are omitted.

To observe the inside of the subject by using the endoscope system 1 of the present embodiment configured as described above, the insertion portion 4 of the endoscope 2 is first inserted into, for example, a body cavity of the subject as in the conventional endoscope system 1.

In this case, the image pickup device 34 generates an image pickup signal based on the subject image of the first region and the subject image of the second region acquired by the image pickup section (objective optical system 40, image pickup device 34) and transmits the image pickup signal to the video processor 32.

An image generation section 32g of the video processor 32 generates image data of the subject from the image pickup signal, the image data including a forward image based on the first subject image and a lateral image based on the second subject image.

The image data of the subject is transmitted to the image processing section 32a of the video processor 32, and the image processing section 32a applies various image processing. That is, the image processing section 32a receives the image data outputted from the image pickup section (objective optical system 40, image pickup device 34) and executes image processing for generating image data (display signal) for display.

The endoscopic image for display generated in this way is transmitted to the display apparatus 35. As a result, the endoscopic image is displayed on a display screen 35a of the display apparatus 35. In this case, the image processing section 32a executes a mode switch control process of selectively switching between a first display mode (first mode) for displaying the images by arranging the lateral images (second field of view images) around the forward image (first field of view image) in a state in which parts of the lateral images (second field of view images) including both sides of the forward image (first field of view image) are continuously connected and a second display mode (second mode) for displaying the images by arranging the lateral images (second field of view images) side by side with the forward image (first field of view image) in a state in which the parts of the lateral images (second field of view images) including both sides of the forward image (first field of view image) are separated and transmitting an image signal for display according to each display mode to the display apparatus 35 (display section). The mode switch control process by the image processing section 32a is executed at an appropriate predetermined timing based on, for example, an instruction from the outside.

The state in which the parts of the lateral images including both sides of the forward image are continuously connected denotes that parts of the lateral images are integrated. The state includes a state in which two lateral images are in contact with each other, a state in which two lateral images are integrated, and a state in which boundary processing is applied to a boundary of the two integrated or contacting lateral images. Examples of the boundary processing include image processing of making the border of the two lateral images inconspicuous and image processing of superimposing a thin boundary line on the border of the two lateral images.

FIGS. 4 and 5 show specific examples of the display form of the endoscope image displayed on the display screen 35a of the display apparatus 35 in the endoscope system 1 of the present embodiment. FIGS. 4 and 5 are diagrams respectively showing display forms of the endoscopic image that can be displayed by the display apparatus 35 in the endoscope system 1 of the present embodiment. Of these, FIG. 4 shows a first display form. FIG. 5 shows a second display form.

First, different images are respectively displayed in two regions (F1, SR1 and SL1) in the rectangular display screen 35a in the first display form of the endoscopic image as shown in FIG. 4. In this case, the rectangular display screen 35a includes, for example, a display region that can display all images based on the image data acquired by the image pickup device 34 and that can also display various information such as patient data. That is, the endoscopic image is displayed by using part of the entire display region of the display screen 35a.

In the example shown in FIG. 4, the region F1 is a region for displaying an object image (image of subject) in the forward field of view generated based on the beam F entering the objective optical system 40 from the forward field of view observation window 12 through the first lens 41. The forward image displayed in the region F1 is displayed, for example, in a substantially circular shape.

The regions SR1 and SL1 are regions for displaying object images (images of subject) in the lateral field of view generated based on the beam S entering the objective optical system 40 from the lateral field of view observation window 13 through the reflective optical system 15. The lateral images of the regions SR1 and SL1 are displayed in a substantially annular shape along a peripheral edge portion of the region F1 of the substantially circular forward image.

Note that an image of part of a substantially right half portion of the lateral field of view observation window 13 is displayed in the region SR1, and an image of part of a substantially left half portion of the lateral field of view observation window 13 is displayed in the region SL1. However, the images of the regions SR1 and SL1 are not actually displayed as independent images, and a continuously connected image without a cut between the respective regions, that is, an integrated image, is displayed.

The region B (region indicated by oblique lines) of FIG. 4 is a region shielded by the support portion 18 and is a region in which the image of the subject is not actually displayed as an endoscopic image. A mask (for example, a black mask) of another part of the display screen 35a may be superimposed on the region B.

The first display form shown in FIG. 4 is a form similar to the endoscopic image displayed in the conventional endoscope system.

On the other hand, the endoscope system 1 of the present embodiment can display the endoscopic image based on the second display form different from the first display form. FIG. 5 shows the second display form of the endoscopic image that can be displayed in the endoscope system 1 of the present embodiment.

In the second display form shown in FIG. 5, a forward field of view image in, for example, a substantially circular shape is displayed in a region F2 at a substantially center portion of the display screen 35a, and left and right lateral images formed in, for example, a substantially trapezoidal shape are respectively displayed side by side in regions on both sides of the region F2. In this case, the forward image displayed in the region F2 of FIG. 5 is an image corresponding to the region F1 of FIG. 4. The respective lateral field of view images displayed in regions SR2 and SL2 of FIG. 5 are images corresponding to the regions SR1 and SL1 of FIG. 4, respectively.

The image generation section 32g of the video processor 32 generates image data of the subject based on the image pickup signal outputted from the image pickup section (objective optical system 40, image pickup device 34). The image processing section 32a receives the image data outputted from the image generation section 32g to generate image data for display.

That is, respective signals from different regions in the image signals based on the image pickup signals from the image pickup device that is the same one image pickup section are used to switch between the first mode and the second mode.

To switch the first display form (first display mode) to the second display form (second display mode), the lateral images are cut from the state in which the regions SR1 and SL1 are arranged around the forward image in the state in which the regions SR1 and SL1 of the lateral images including both sides of the forward image (region F1) are continuously connected, that is, partially in contact with each other. The parts of the lateral images including both sides of the forward image are separated, and the positions of the lateral images are changed as necessary to change the manner of lining up the images. The lateral images are arranged side by side with the forward image.

To cut the lateral images, there are a method of breaking down the continuously connected lateral images to separate the respective lateral images and arranging the lateral images side by side with the forward image and a method of extracting parts of the continuously connected lateral images to separate the respective lateral images and arranging the lateral images side by side with the forward image.

For example, to prevent both regions near the boundary of the regions SR1 and SL1 of FIG. 4 from being displayed on upper edges of the separately arranged regions SR2 and SL2 of FIG. 5, the image processing section 32a may execute image processing of partially cutting off parts near the boundary of the regions SR1 and SL1 of FIG. 4 to create images of the regions SR2 and SL2 to display the images in the second mode when the first mode is shifted to the second mode.

In this case, the image processing section 32a may execute, for example, image processing corresponding to a plurality of display forms all the time and continue generating a plurality of image data corresponding to the plurality of display forms. In this case, the output of the image data to the display apparatus 35 is switched according to, for example, an instruction from the outside generated by operation by the user or according to an operation form or the like of the endoscope system 1.

The display apparatus 35 receives the image signal from the image processing section 32a and displays, based on one of the first display mode and the second display mode, endoscopic images respectively based on the forward field of view image (first field of view image) and the lateral field of view images (second field of view images).

Other than the configuration, the image processing section 32a may be configured to, for example, continue generating image data corresponding to the display form of one of the first display form of FIG. 4 and the second display form of FIG. 5 and output the image data to the display apparatus 35 in a normal case. In this case, when a display switch instruction signal is generated by the user for example, the image processing by the image processing section 32a can be controlled, in response to the signal, to switch the process corresponding to the display form being displayed to a process corresponding to another display form at the reception timing.

In this way, a plurality of display forms of the endoscopic image displayed on the display screen 35a of the display apparatus 35 are prepared in the endoscope system 1 of the present embodiment. An endoscopic image in a display form desired by the user among the plurality of prepared display forms or in an appropriate display form according to the operation form of the endoscope system 1 is selectively displayed on the display screen 35a of the display apparatus 35.

For example, when the endoscope system 1 is in use, the user can perform switching operation at a desired timing at a desired time to switch the display form being displayed on the display apparatus 35. In this case, the scope switch 25 can be used to perform the switching operation of the display form, for example. Other than the operation, the keyboard 36 that is an external input device or a foot switch, a pointing device, a touch panel, or the like may be used to perform the operation. An instruction signal generated from the scope switch 25 or the external input device (keyboard 36 or a foot switch, a pointing device, a touch panel, or the like) is detected by the operation detection section 32b of the video processor 32. In response to the instruction signal, the video processor 32 controls the image processing section 32a to switch and control necessary image processing or to switch and control the image data outputted to the display apparatus 35. In this way, the image data according to the display form desired by the user is transmitted to the display apparatus 35 and displayed on the display screen 35a of the display apparatus 35.

Other than this, a program may be configured for the display in an appropriate display form according to the operation form of the endoscope system 1. More specifically, for example, a display form can be provided to mainly include the forward field of view image in the insertion direction during the insertion operation of the insertion portion 4 of the endoscope 2, and a display form can be provided to allow always observing the lateral field of view images along with the forward field of view image to allow observing a wide range when an abnormal part in the body cavity is searched. Such a switch can be automatically made according to the operation form.

The image processing section 32a executes the process of switching the display form according to a predetermined instruction. A form of the switch display process may be, for example, a switching process of instantaneously switching the display of the display screen 35a being displayed according to the switching instruction or may be a switching process form with a display effect of switching the display by gradually fading out the display of the display screen 35a being displayed and gradually fading in the display to be displayed.

Here, a case of switching the first display form of FIG. 4 to the second display form of FIG. 5 will be considered, for example. In this case, the left and right lateral images are deformed from the respective arc-shaped images of the regions SR1 and SL1 of FIG. 4 to the respective trapezoidal images of the regions SR2 and SL2 of FIG. 5. When the shape of the images is deformed, the images can be shown and displayed by gradually changing and breaking down the shape using animation, for example.

That is, the image processing section 32a executes the switch control process of the first display form (first display mode) and the second display form (second display mode) based on the setting of one of the setting in which the first display form (first display mode) and the second display form (second display mode) are gradually switched and the setting in which the first display form (first display mode) and the second display form (second display mode) are instantaneously switched. The image processing section 32a displays the images in the corresponding display form on the display screen 35a of the display apparatus 35.

As described, according to the first embodiment, the endoscope system 1 realizes the display form of the endoscopic image displayed by using the display apparatus 35 not only by the display form (first display form) as in the conventional system, but also by the display form (second display form) that is a different form. The endoscope system 1 is configured to switch the plurality of display forms at a timing desired by the user or to automatically switch the plurality of display forms according to the operation form.

According to the configuration, when the endoscope system 1 is in use, the user can select an appropriate display form at a desired time. This can realize an endoscope system that can be more easily used.

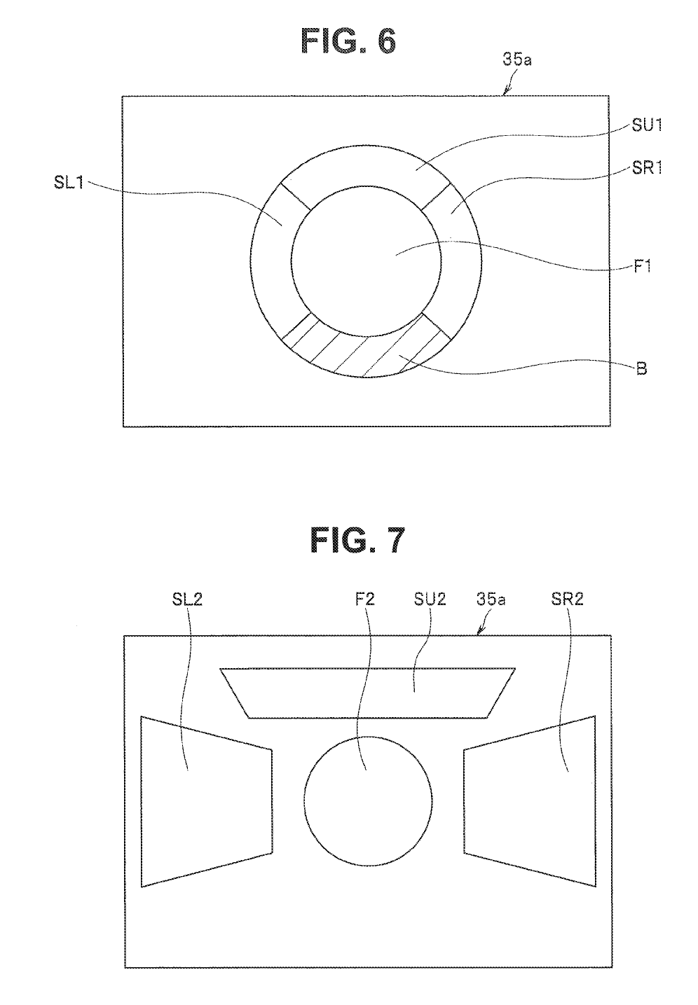

Although the first display shown form in FIG. 4 and the second display form shown in FIG. 5 are illustrated in the first embodiment, the display form of the endoscopic image that can be realized in the present endoscope system 1 is not limited to the examples of display illustrated in FIGS. 4 and 5. For example, display forms as shown in FIGS. 6 and 7 can also be considered.

FIGS. 6 and 7 are diagrams showing modifications of the display form of the endoscopic image that can be displayed by the display apparatus in the endoscopic system of the first embodiment of the present invention. Of these, FIG. 6 shows a modification of the first display form. FIG. 7 shows a modification of the second display form.

The display of the modification of the first display form shown in FIG. 6 is substantially the same as the first display form of FIG. 4. That is, the forward field of view image of the region F1 in the display screen 35a is displayed in a substantially circular shape, and the lateral field of view images are displayed in a substantially annular shape in regions (SR1, SU1, and SL1) on the peripheral edge portion of the region F1. In this case, the substantially annular lateral field of view images include three regions SR1, SU1, and SL1 in the example shown in FIG. 6, and an image of part of a substantially right half portion of the lateral field of view observation window 13 is displayed in the region SR1. An image of part of a substantially upper half portion of the lateral field of view observation window 13 is displayed in the region SU1, and an image of part of a substantially left half portion of the lateral field of view observation window 13 is displayed in the region SL1. In this case, the images of the respective regions SR1, SU1, and SL1 are not independent and are displayed as one continuous image. The region B (region indicated by oblique lines) is a region shielded by the support portion 18 as in the display form of FIG. 4 and is a non-display region that is a non-display part in which the image is not displayed.

In other words, the non-display part is a part in which part of the lateral field of view images (lateral images, second images) is not displayed in the state in which the lateral field of view images (lateral images, second images) are displayed around the forward field of view image (forward image, first image) in the first display form (first display mode). A mask (for example, a black mask) of another part of the display screen 35a may be superimposed on the region B.

In accordance with this, in the modification of the second display form of FIG. 7, the substantially circular forward field of view image corresponding to the region F1 of FIG. 6 is displayed in the region F2 on the substantially center portion of the display screen 35a, and the lateral field of view images respectively corresponding to the regions SR1 and SL1 of FIG. 6 are respectively displayed in the regions SR2 and SL2 on both left and right sides of the region F2 as in the second display form of FIG. 5. In addition, an upper field of view image corresponding to the region SU1 of FIG. 6 is displayed in an upper region SU2 of the region F2 of FIG. 7 in the present modification.

That is, when the display is switched and shifted from the first display form (first display mode) of FIG. 6 to the second display form (second display mode) of FIG. 7 in the modification of the display form, the image processing section 32a arranges the images of the lateral field of view images (lateral images, second images) in the regions SR2 and SL2 side by side with both sides of the region F2 of the forward field of view image (forward image, first image) and arranges the image of the lateral field of view images (lateral images, second images) in the region SU2 adjacent to a side (for example, upper part) different from both sides of the region F2 of the forward field of view image (forward image, first image).

In this way, the display in the first display form substantially the same as for the conventionally general and normal display endoscopic image and the display in the second display form different from the first display form can be appropriately switched in the endoscope system 1 of the first embodiment.

Here, various operations regarding the image display of the images displayed on the display screen 35a may be able to be further performed in the endoscope system 1 of the first embodiment according to the operation by the user, such as predetermined modification like changing the sizes of individual images (contraction and expansion operation) or changing the display positions of individual images (movement operation, rotation operation), correction of the shapes of the images, and setting of display/non-display of desired images. The image processing section 32a executes control processing of instructions of the operations according to the operation of the external input device by the user.

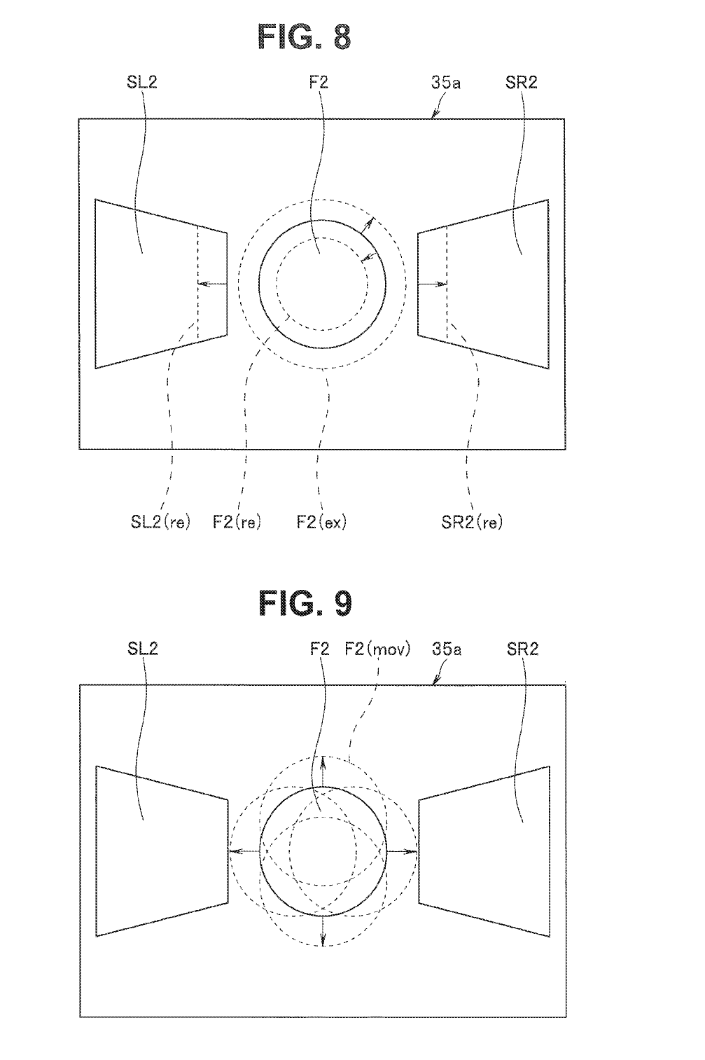

More specifically, FIG. 8 shows an example of display when expansion operation or contraction operation of the images of the respective display regions F2, SR2, and SL2 is performed in the display screen 35a in which the images are displayed in the second display form (FIG. 5), for example. The display indicated by solid lines in FIG. 8 shows the images of the respective regions F2, SR2, and SL2 in the normal second display form. Here, the user uses the external input device, such as a touch panel, to perform slide operation or the like in arrow directions in FIG. 8 so that the solid line display of FIG. 8 is switched to dotted line displays F2 (re), F2 (ex), SR2 (re), SL2 (re) and the like. As a result, the images of the respective regions F2, SR2, and SL2 of FIG. 8 are expanded or contracted and displayed according to the operation. In this case, although the individual images in the respective regions are expanded or contracted image by image in the example, there can be an operation example other than the example. For example, a partial region in the respective regions can be selected, and only the selected region can be expanded and displayed. Specifically, when a lesion part or the like is discovered in a partial region in the forward field of view image of the region F2 for example, expanding and displaying only the partial region including the lesion part can contribute to the discovery of a specific lesion part.

Here, the image processing section 32a expands or contracts the forward field of view image (forward image, first image) and the lateral field of view images (lateral images, second images) in the second display form (second display mode) to adjust the size relationship between the forward field of view image (forward image, first image) and the lateral field of view images (lateral images, second images) to display the images on the display screen 35a of the display apparatus 35.

The second display form and the modification shown in FIGS. 5 and 7 are imaged and described such as to independently display the images of the respective corresponding regions. In this case, the continuity between the respective regions is lost, and the display may be hard to view. Therefore, as shown for example in FIG. 9, the external input device, such as a touch panel, is used to move parallel the forward field of view image of the region F2 (can also be another region) on the screen, from the solid line display of FIG. 9 to an arbitrary position as indicated by a dotted line display F2 (mov). As a result, when the regions F2 and SR2 are displayed adjacent to each other for example, the images of both region F2 and the region SR2 can be displayed as substantially continuous images. Therefore, when there is a lesion part across both regions, the lesion part can be displayed and observed better.

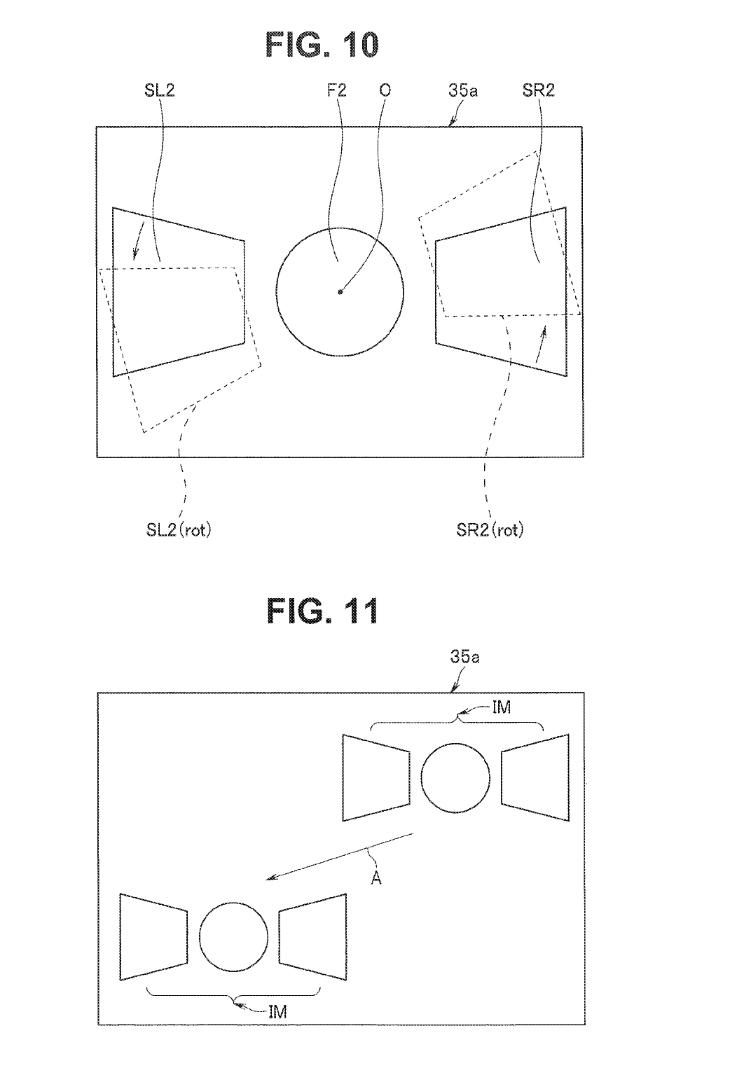

Furthermore, depending on the insertion state of the endoscope 2 inserted into the body cavity for example, the images displayed on the display screen 35a of the display apparatus 35 may be displayed at angles with which the images are hard to view. Therefore, a set of respective regions F2, SR2, and SL2 of the endoscopic image may be able to be rotated and moved by setting, for example, a center point O of the forward field of view image of the region F2 as a rotation center, so that dotted line displays SR2 (rot) and SL2 (rot) are provided. In this case, although the position of the region F2 is not changed as shown in FIG. 10, the image in the region F2 is rotated and moved. Therefore, the endoscopic image can be changed and set at a position where the user can easily view the endoscopic image.

Here, the image processing section 32a moves parallel or rotates and moves the forward field of view image (forward image, first image) and the lateral field of view images (lateral images, second images) in the second display form (second display mode) to adjust the positional relationship between the forward field of view image (forward image, first image) and the lateral field of view images (lateral images, second images) and display the images on the display screen 35a of the display apparatus 35.

On the other hand, a large high-resolution display screen 35a of the display apparatus 35 is more inexpensively provided in recent years. When the display apparatus 35 with the large display screen 35a is adopted, more information can be displayed on one display screen 35a at the same time, and this is convenient for the user. In this case, even if the endoscopic image displayed on the enlarged display screen 35a becomes relatively small, an endoscopic image with a resolution equivalent to the conventional small display screen 35a can be maintained. Therefore, the user may be able to arbitrarily set the display position of an endoscopic image IM in the entire display region of the display screen 35a as shown in FIG. 11.

Other than the operation described above, a correction process for the image in a designated region in the endoscopic image, such as a distortion correction process of the image displayed in a substantially circular shape using a wide-angle lens and a changing process of the display shape (deformation process of deforming the circular image into a rectangular image or the like), may be executed according to desired designation operation by the user as shown for example in FIG. 18.

More specifically, the image processing section 32a executes an image signal conversion process of hiding part of the forward field of view image (forward image, first image) and the lateral field of view images (lateral images, second images) such that the displayed images become rectangular in the second display form (second display mode), for example.

The image processing section 32a also executes a correction process of removing the distortion generated around the forward field of view image (forward image, first image) or the lateral field of view images (lateral images, second images) and displays the corrected images on the display screen 35a of the display apparatus 35.

Furthermore, when the first display form (first display mode) is shifted to the second display form (second display mode), a process of changing the degree of distortion of the lateral images between the first display form (first display mode) and the second display form (second display mode) may be executed.

For example, a rate of expanding the length of the part adjacent to the forward image and the rate of expanding the length of the part on the side away from the forward image may be changed in the lateral images. In the state in which the parts of the lateral images including both sides of the forward image are continuously connected, the length of the lateral images in the up and down direction may be compressed more than in the state in which the lateral images are arranged around the forward image, and the length in the left and right direction may be extended. In this case, the degree of deformation of the lateral images in the up and down direction is greater than the degree of deformation of the lateral images in the left and right direction.

In addition, the user can select and operate a desired display region, such as a display region including a lesion part, of three or four display regions displayed in the display screen 35a, for example. Only the selected region may be displayed, and the images in the other non-selected regions may be put into a non-display state. Such a display control is also possible. In this case, the selected and displayed image may be able to be further expanded and displayed, for example.

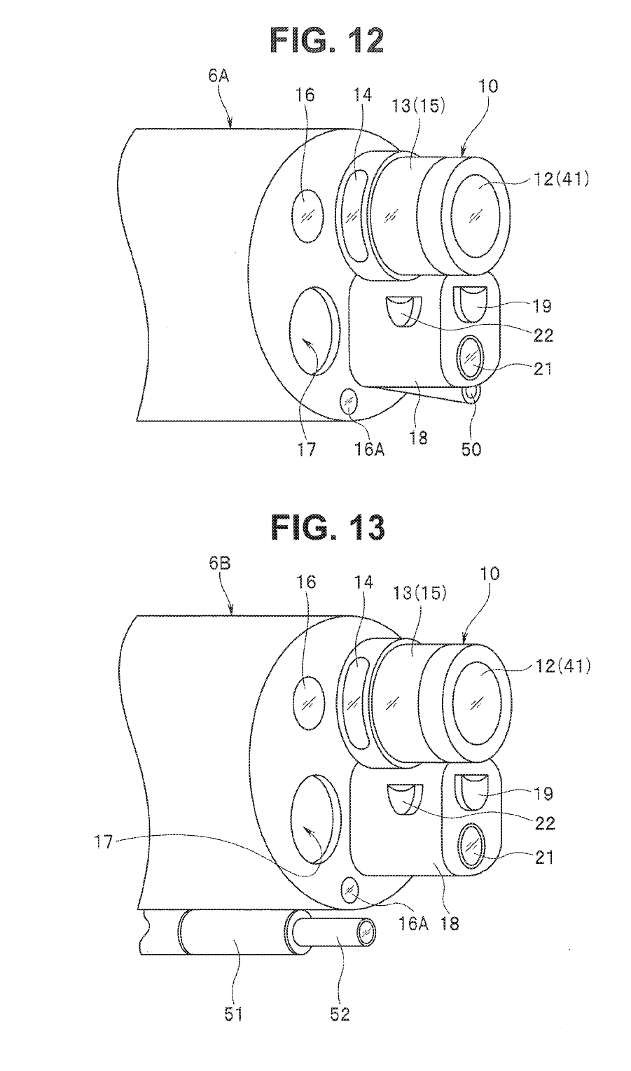

On the other hand, in the endoscope system 1 of the first embodiment, the region B of part of the lateral field of view images is shielded by the support portion 18 as shown in FIGS. 4 and 6 as described above, and there is a non-display region that is a non-display part. Examples of constitutive schemes for eliminating the non-display region include modifications respectively illustrated in FIGS. 12, 13, and the like.

FIG. 12 is an external perspective view showing a first modification of the distal end portion of the endoscope insertion portion in the endoscope system of the first embodiment of the present invention. FIG. 13 is an external perspective view showing a second modification of the distal end portion of the endoscope insertion portion in the endoscope system of the first embodiment of the present invention.

The first modification of FIG. 12 and the second modification of FIG. 13 further include a third optical system that can acquire an image of a region corresponding to the non-display region B in the endoscope system of the first embodiment.

More specifically, a third optical system 50 is located on an outer edge portion of the support portion 18 provided on a distal end portion 6A of the endoscope insertion portion in the first modification shown in FIG. 12. The third optical system 50 includes an optical system that can form a lateral field of view image of a lower side of the support portion 18 of the lateral field of view image, on the light receiving surface of the image pickup device 34. The third optical system 50 is made of an optical system that can form a wide field of view image capable of covering the lateral field of view image in addition to the forward field of view image. In other words, the third optical system 50 is a third subject image acquisition portion provided on the distal end portion 6A of the insertion portion 4 and configured to acquire a third field of view image from a third direction different from each of the first direction facing the forward field of view and the second direction facing the lateral field of view. Here, the third field of view image from the third direction is, for example, a lower field of view image from the field of view of part of the lateral field of view image facing the lateral field of view, particularly the field of view closer to the lower side, as described above.

Since the third optical system 50 is newly located on the insertion portion 4, a third illumination window 16A for illuminating the region covered by the third optical system 50 is provided on a distal end surface of the distal end portion 6A. On the third illumination window 16A, the distal end surface of the light guide cable extended from the light source apparatus 31 is arranged in the distal end portion 6A, as in the other illumination windows. The other components are the same as in the first embodiment.

A third optical system 52 in a form different from the first modification is located in the second modification shown in FIG. 13. The third optical system 52 is inserted into an external channel 51 integrally located on a peripheral surface of a distal end portion 6B of the endoscope insertion portion and is protruded forward from a distal end of the distal end portion 6B.

As in the first modification, the third optical system 52 is also a subject image acquisition portion including a wide field optical system that can form a lateral field of view image of the lower side of the support portion 18 of the lateral field of view image on the light receiving surface of the image pickup device 34. In the present modification, the third illumination window 16A for illuminating the region covered by the third optical system 52 is also provided on a distal end surface of the distal end portion 6B. The configuration of the third illumination window 16A is the same as in the first modification. The other components are the same as in the first embodiment.

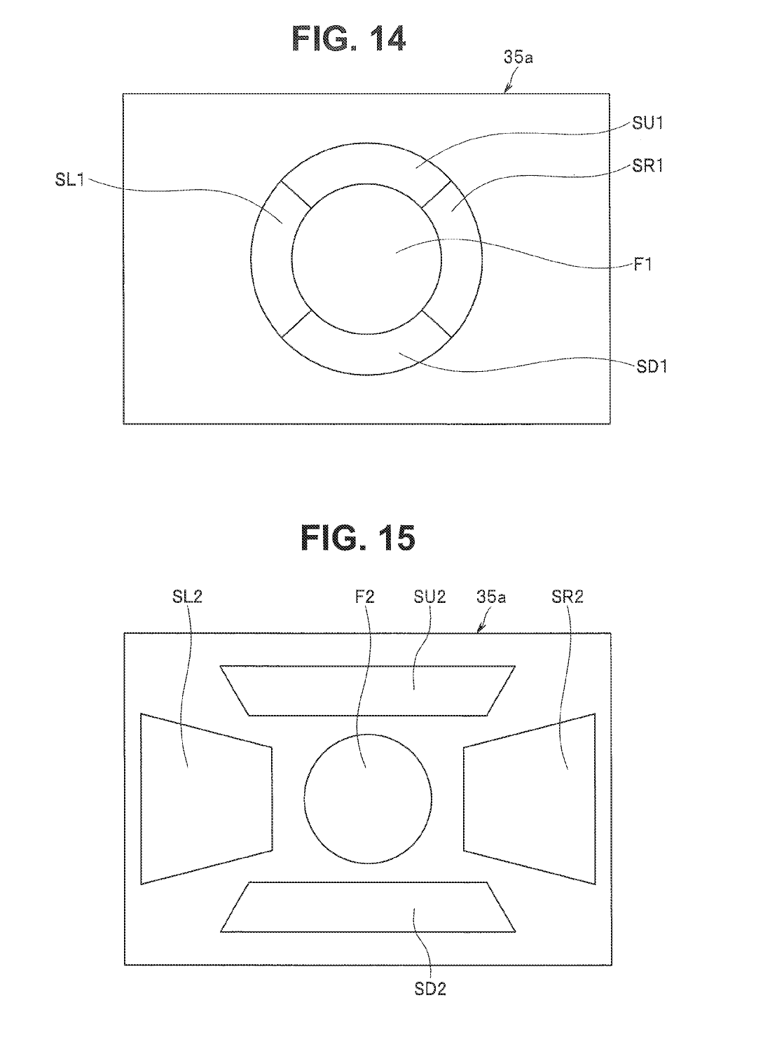

In the endoscope system including the distal end portions 6A and 6B of the first modification and the second modification configured in this way, the endoscopic images displayed on the display screen 35a of the display apparatus 35 are, for example, as shown in FIGS. 14 and 15.

FIGS. 14 and 15 are diagrams showing examples of the display form of the endoscopic image displayed on the display screen of the display apparatus in the endoscope system including the distal end portion of the endoscope insertion portion of each modification shown in FIGS. 12 and 13. Of these, FIG. 14 illustrates the first display form. FIG. 15 illustrates the second display form.

The display of the first display form shown in FIG. 14 is substantially the same as the first display form shown in FIG. 4 described in the first embodiment or shown in FIG. 6 in the modification of the first embodiment. That is, the forward field of view image of the region F1 in the display screen 35a is displayed in a substantially circular shape, and the lateral field of view images are displayed in a substantially annular shape in regions (SR1, SL1, SU1, and SD1) of the peripheral edge portion of the region F1. In this case, the example shown in FIG. 14 is different in that the substantially annular lateral field of view images include four regions SR1, SL1, SU1, and SD1 and that an image of part of a substantially lower half portion of the distal end portions 6A and 6B of the images formed by the third optical systems 50 and 52 is displayed in the region SD1 corresponding to the non-display region B in the first display form of FIGS. 4 and 6. In this case, the images of the respective regions SR1, SL1, SU1, and SD1 are not independent and are displayed as one continuous image.

In accordance with this, the substantially circular forward field of view image corresponding to the region F1 of FIG. 14 is displayed in the region F2 on the substantially center portion of the display screen 35a in the second display form of FIG. 15, as in the second display form of FIGS. 5 and 7. The lateral field of view images respectively corresponding to the regions SR1 and SL1 of FIG. 14 are respectively displayed on the regions SR2 and SL2 on both left and right sides of the region F2. An upper field of view image corresponding to the region SU1 of FIG. 14 is displayed in the upper region SU2 of the region F2 of FIG. 15, and a lower field of view image corresponding to the region SD1 of FIG. 14 is displayed in a lower region SD2 of the region F2 of FIG. 15.

That is, when the display is switched and shifted from the first display form (first display mode) of FIG. 14 to the second display form (second display mode) of FIG. 15 in the example of the display form, the image processing section 32a arranges the images of the lateral field of view images (lateral images, second images) in the regions SR2 and SL2 side by side with both sides of the region F2 of the forward field of view image (forward image, first image) and arranges the images of the lateral field of view images (lateral images, second images) in the regions SU2 and SD2 adjacent to both sides of the forward field of view image (forward image, first image) on sides different from both sides of the region F2 of the forward field of view image (forward image, first image).

In this way, the third optical systems 50 and 52 can be further provided to acquire the images of substantially the whole circumference for the lateral field of view images, and an endoscopic image of a wider range can be observed in the first and second modifications shown in FIGS. 12 and 13.

In this case, the image data acquired by the third optical systems 50 and 52 is used for the image displayed in the region SD2 in the second display form (second display mode) as described above. More specifically, the third field of view image (lower field of view image) from the third direction (lower field of view) different from each of the first direction facing the forward field of view and the second direction facing the left and right lateral field of views and the upper field of view of the lateral field of view is superimposed (that is, placed on top of each other) and displayed, for example.

Other than this, the image processing section 32a may also perform control to make a switch to a third display form (third display mode; third mode) for selecting and displaying, on the display screen 35a of the display apparatus 35, only the endoscopic image based on the third field of view image formed from the image data generated by the image generation section 32g based on the image pickup signal acquired by the third optical systems 50 and 52.

By the way, the treatment instrument channel is inserted into the flexible tube portion 8 from the treatment instrument insertion port 27 of the operation portion 3 of the endoscope 2 to the channel distal end opening portion 17 of the distal end portion 6 in the endoscope system of the first embodiment as described above. According to the configuration, when the treatment instrument is inserted from the treatment instrument insertion port 27, the treatment instrument goes through the treatment instrument channel, and then the distal end section of the treatment instrument protrudes from the channel distal end opening portion 17. According to the configuration, the distal end part of the treatment instrument can be caused to reach a desired part to be inspected inside of the body cavity from the outside of the body cavity to perform various treatments such as therapy.

In this case, part of the distal end of the treatment instrument inserted into the treatment instrument channel of the insertion portion 4 is displayed in the endoscopic image. When the first display form (first display mode) and the second display form (second display mode) are switched in this state, the treatment instrument may not be displayed in one of the display modes.

However, when the treatment instrument is used to perform treatment or the like while using the endoscope system to observe the endoscopic image, it is desirable that the position of the treatment instrument, the movement trajectory of the treatment instrument, and the like are always displayed in the endoscopic image being displayed. Therefore, another modification regarding the distal end portion of the endoscope insertion portion in the endoscope system of the first embodiment illustrated below shows a configuration that can display the movement trajectory of the treatment instrument in the endoscopic image.

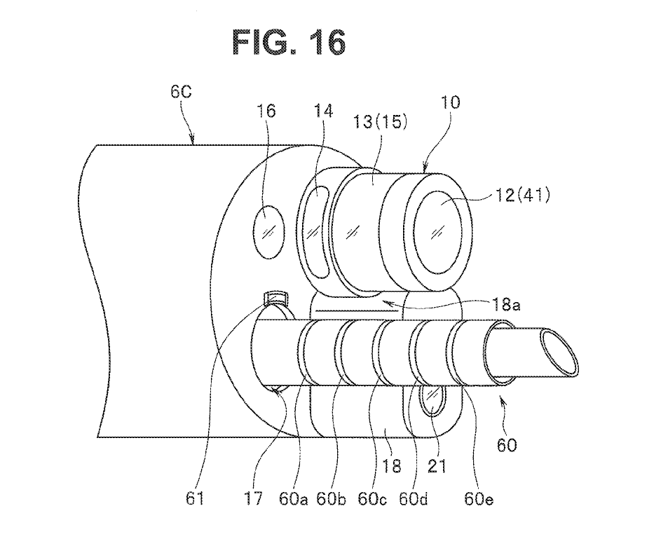

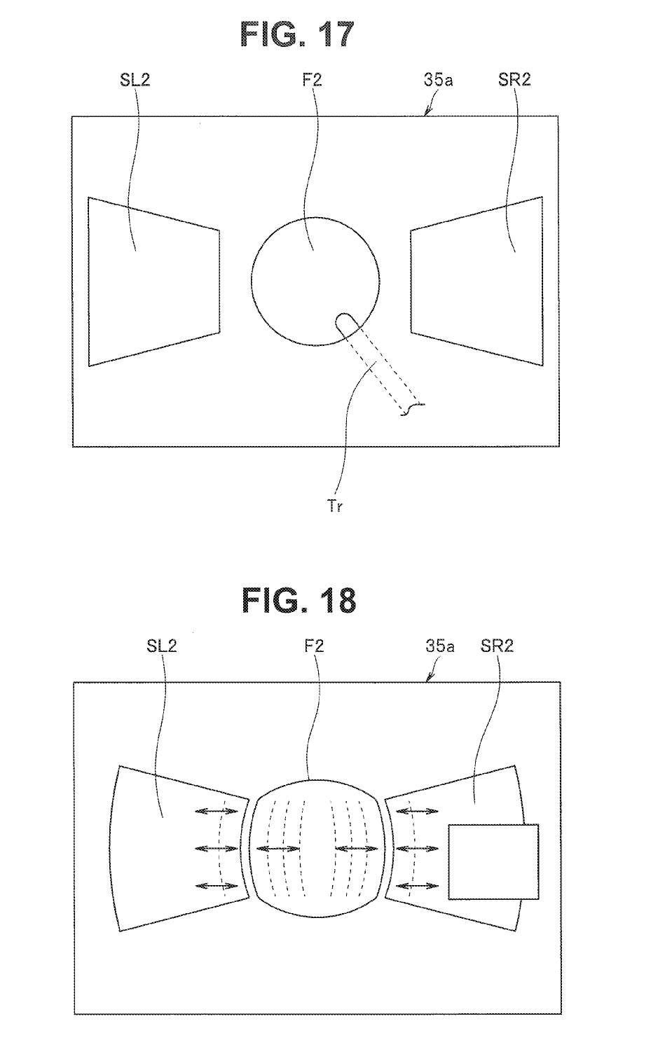

FIG. 16 is an external perspective view showing another modification of the distal end portion of the endoscope insertion portion in the endoscope system of the first embodiment of the present invention. FIG. 17 is a diagram showing an example of display form of the endoscopic image displayed on the display screen of the display apparatus in the endoscope system of FIG. 16.

As shown in FIG. 16, a detection sensor 61 configured to detect the position of a distal end part of a treatment instrument 60 is located near the channel distal end opening portion 17 formed on the distal end surface in a distal end portion 6C of the endoscope according to the other modification. Various forms using an infrared sensor or the like can be applied as the detection sensor 61. The detection sensor 61 is under the control of, for example, the video processor 32, and a detection signal of the detection sensor 61 is transmitted to the video processor 32.

On the other hand, a plurality of markers 60a, 60b, 60c, 60d, 60e . . . for indicating specific parts of the treatment instrument 60 are provided on a plurality of places closer to the distal end portion of the treatment instrument 60 used in the endoscope system including the distal end portion 6C of the other modification as shown in FIG. 16. Here, each of the markers 60a, 60b, 60c, 60d, 60e . . . is in a form that allows individually specifying the marker. For example, a different color arrangement is set for each of the markers 60a, 60b, 60c, 60d, 60e . . . Other than specifying the markers based on color information, each of the markers 60a, 60b, 60c, 60d, 60e . . . may be formed by a circumferential groove or a circumferential projection portion, and the groove width or the width of the projection portion may vary in each of the markers 60a, 60b, 60c, 60d, 60e . . . to allow specifying the individual markers.

In this way, when the detection sensor 61 detects each of the markers 60a, 60b, 60c, 60d, 60e . . . , the video processor 32 receives the detection results and executes a control process of controlling the image processing section 32a to draw information, such as the movement trajectory of the distal end portion of the treatment instrument 60, in the endoscopic image. As a result, an endoscopic image as shown for example in FIG. 17 is displayed on the display screen 35a of the display apparatus 35. The example of display shown in FIG. 17 is equivalent to the second display form of FIG. 5 described in the first embodiment. A trajectory Tr of the treatment instrument 60 generated by the image processing section 32a is displayed in the endoscopic image.

Note that in the endoscope system of the first embodiment, an identification value, such as a doctor ID, can be inputted to the video processor 32, and a display method requested for each preferred display initial setting and each type of operative method of each surgeon among the display methods described in the first embodiment can be recorded. The identification value may be able to be inputted in the next use to call the recorded display form with the same identification value.

At least one of the endoscopic images in the display forms described in the first embodiment can be designated to save a still image or a movie in a recording section 38 (see FIG. 1) connected to or embedded in the video processor 32. In such a case, the following saving methods of the endoscopic image can be considered:

A method of saving the image in one of the first display mode and the second display mode that is the same display mode as the display form of the display apparatus 35;

A method of saving the movie in one of the first display mode and the second display mode regardless of the display form of the display apparatus 35;

A method in which the image processing section 32a creates endoscopic images in the first display mode and the second display mode at the same time, and both endoscopic images in the first display mode and the second display mode are saved regardless of the display form of the display apparatus 35 (note that in this case, the endoscopic images are synchronized between the modes in chronological order).

The image processing section 32a may call up and process an endoscopic image saved in the recording section 38 in the first display mode to newly create an endoscopic image in the form of the second display mode and may call up and process a movie saved in the recording section 38 in the second display mode to newly create endoscopic images in the form of the first display mode.

The image processing section 32a may use an index, such as an icon, to display a correspondence between the first display mode and the second display mode by indicating a part in the second display mode corresponding to a part designated by the user in the endoscopic image displayed in the first display mode or by indicating a part in the first display mode corresponding to a part designated by the user in the endoscopic image displayed in the second display mode.

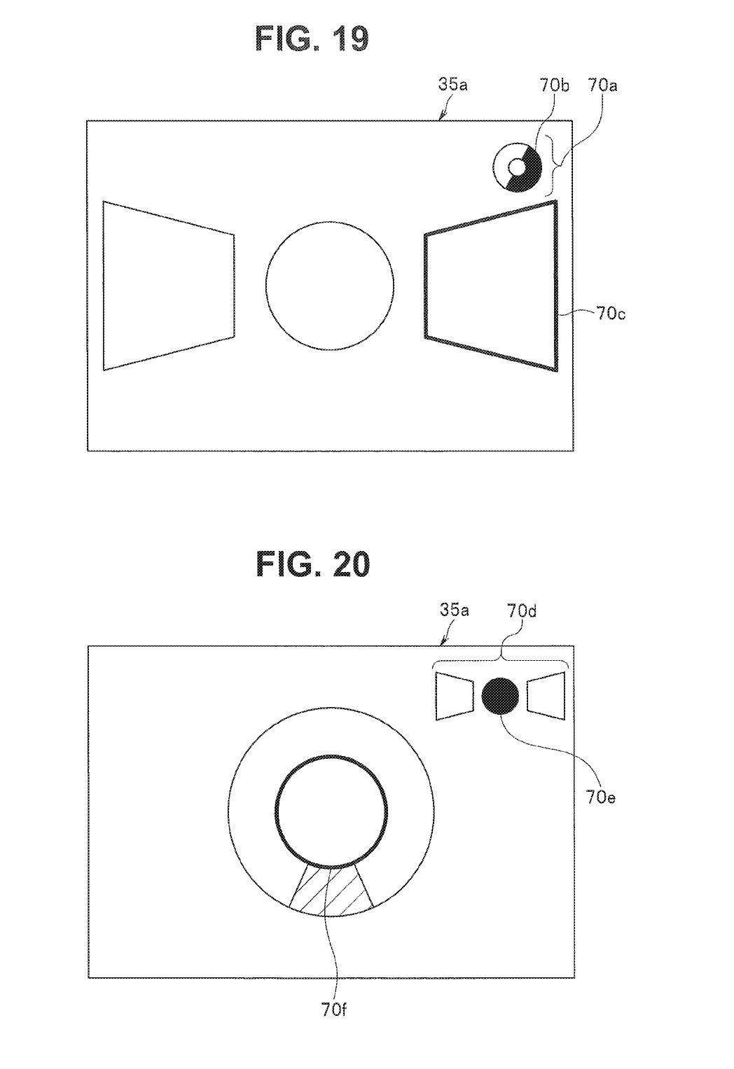

More specifically, as shown for example in FIGS. 19 and 20, icons 70a and 70d simulating the display form in the case of the first (second) display mode can be displayed in a partial region of the display screen 35a of the display apparatus 35 in the second (first) display mode. Identifications can be displayed to allow easily identifying regions (reference signs 70c and 70f) respectively corresponding to regions (see reference signs 70b and 70e) of field of view images in the icons.

Next, a second embodiment of the present invention will be described with reference to an illustrated embodiment. Scaling of each constituent element may vary in each drawing used in the following description in order to illustrate each constituent element in a size that allows recognizing the constituent element on the drawing. Therefore, quantities of the constituent elements described in the drawings, shapes of the constituent elements, ratios of sizes of the constituent elements, and relative positional relationships between respective constituent elements of the present invention are not limited only to the illustrated modes.

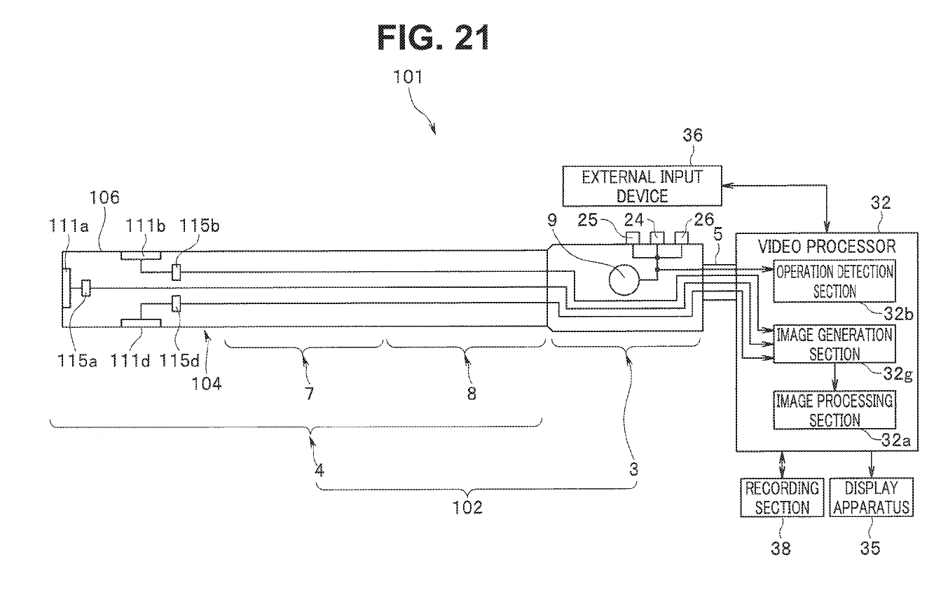

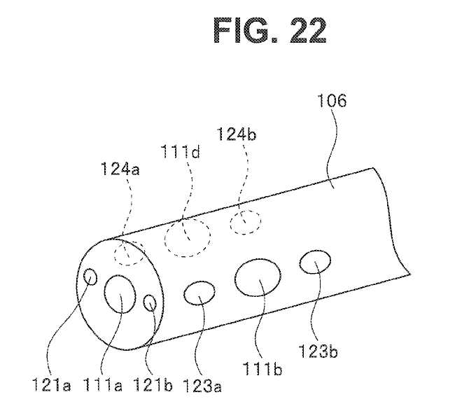

FIGS. 21 and 22 are diagrams showing schematic configurations regarding an endoscope system of the second embodiment of the present invention.

First, an overall configuration of the endoscope system of the second embodiment of the present invention will be simply described by mainly using FIGS. 21 and 22. Note that parts different from the first embodiment will be mainly described, and parts with common configurations will not be described.

An endoscope system 101 of the present embodiment includes an endoscope 102, the video processor 32, the display apparatus 35, the external input device 36 such as a keyboard, and the like.

The endoscope 102 includes the operation portion 3, the insertion portion 4, the universal cord 5, and the like. Among these, the insertion portion 4 is an elongated tubular constituent unit formed by consecutively connecting the distal end portion 6, the bending portion 7, and the flexible tube portion 8 in the order from a distal end. The proximal end of the insertion portion 4 is consecutively connected to the distal end of the operation portion 3. The insertion portion 4 is a constituent portion inserted into a lumen, that is, a body cavity, of the subject during the use of the endoscope 2.

The operation portion 3, the insertion portion 4, the bending portion 7, the flexible tube portion 8, the bending operation knob 9, the air/liquid feeding operation button 24, the scope switch 25, the suction operation button 26, the connector 29, the video processor 32, the connection cable 33, the display apparatus 35, the external input device 36, and other components are the same as in the first embodiment. The components not described above have the same configurations as in a conventionally and generally implemented endoscope system.

Note that the configuration of the distal end portion 6 of the endoscope 102 of the present embodiment is mainly different from the first embodiment. A detailed configuration of the distal end portion 6 will be described by mainly using FIGS. 21 and 22.

A forward field of view observation window 111a for observing a front-view direction (first direction) including the forward direction substantially parallel to the longitudinal direction of the insertion portion 4 is arranged on the distal end surface of the distal end portion 6 of the endoscope 102.

Here, the forward field of view observation window 111a functions as a first subject image acquisition portion configured to acquire a first subject image (will be called a forward subject image or a first subject image) that is a forward field of view image from the forward region including the forward direction of the insertion portion that is a first direction. That is, the first subject image is a field of view image of a first region including the forward direction substantially parallel to the longitudinal direction of the insertion portion 4.

The first subject image acquisition portion is a forward subject image acquisition portion configured to acquire a field of view image of the region including the forward direction of the insertion portion 4. Note that the forward field of view observation window 111a as a first subject image acquisition portion is arranged in a direction in which the insertion portion 4 is inserted into the longitudinal direction distal end portion of the distal end portion 6 of the insertion portion 4.

An image pickup section 115a also configuring the forward subject image acquisition portion for picking up a subject image acquired by the forward field of view observation window 111a is provided inside of the forward field of view observation window 111a configuring the forward subject image acquisition portion.

A plurality of side-view observation windows 111b and 111d for observing the lateral field of view from lateral regions including a side-view direction (second direction) including a direction intersecting the longitudinal direction of the insertion portion 4 that is at least partially different from the front-view direction (first direction), that is, including the lateral direction of the insertion portion 4, are arranged on the side surface of the distal end portion 6 of the endoscope 102.

The lateral field of view observation windows 111b and 111d function as a second subject image acquisition portion configured to acquire second subject images (will be called lateral subject images or second subject images) that are lateral field of view images from lateral regions including the lateral direction of the insertion portion 4 that is a second direction. That is, the second subject images are field of view images of a second region including the lateral direction of the insertion portion 4 that is the radial direction of the insertion portion 4, that is, a direction inclined relative to the longitudinal direction of the insertion portion 4 (for example, substantially perpendicular direction of the longitudinal direction of the insertion portion 4).

Note that the lateral region (first region) and the forward region (first region) are at least partially different regions, and part of the region of the lateral region (first region) may or may not overlap with the forward region (first region).

The second subject image acquisition portion is a lateral subject image acquisition portion configured to acquire the field of view images of the regions including the lateral directions of the insertion portion 4. Note that the lateral field of view observation windows 111b and 111d that are the second subject image acquisition portions are arranged in the radial direction of the distal end portion 6 of the insertion portion 4, at uniform intervals of 180 degrees in the circumferential direction of the distal end portion 6, for example.

An image pickup section 115b also configuring a lateral subject image acquisition portion configured to pick up a subject image acquired by the lateral field of view observation window 111b is provided inside of the lateral field of view observation window 111b configuring the lateral subject image acquisition portion. An image pickup section 115d also configuring the lateral subject image acquisition portion configured to pick up the subject image acquired by the lateral field of view observation window 111d is provided inside of the lateral field of view observation window 111d configuring the lateral subject image acquisition portion.

That is, separate image pickup sections are respectively provided inside of the individual observation windows in the present embodiment.

Note that the number of lateral field of view observation windows 111b and 111d arranged at uniform intervals in the circumferential direction of the distal end portion 6 is not limited to two, and a plurality of another number of lateral field of view observation windows may be arranged.

On the distal end surface of the distal end portion 6 of the endoscope 2, forward observation windows 121a and 121b for emitting illumination light to a range of the field of view of the forward field of view observation window 111a is arranged on positions adjacent to the forward field of view observation window 111a. On the side surface of the distal end portion 6 of the endoscope 2, side-view illumination windows 123a and 123b for emitting illumination light to a range of the field of view of the side-view observation window 111b are arranged at positions adjacent to the side-view observation window 111b. On the side surface of the distal end portion 6 of the endoscope 2, side-view illumination windows 124a and 124b for emitting illumination light to a range of the field of view of the side-view observation window 111d are arranged on positions adjacent to the side-view observation window 111d.

Based on the subject image of the first region acquired by the forward subject image acquisition portion (forward field of view observation window 111a, image pickup section 115a) and the subject images of the second regions acquired by the lateral subject image acquisition portion (lateral field of view observation window 111b, image pickup section 115b) and the other lateral subject image acquisition portion (lateral field of view observation window 111d, image pickup section 115d), the image pickup device 34 generates an image pickup signal and transmits the image pickup signal to the video processor 32.

From the image pickup signal, the image generation section 32g of the video processor 32 generates image data of the subject including the forward image based on the first subject image and the lateral images based on the second subject images.

The image data of the subject is transmitted to the image processing section 32a of the video processor 32, and the image processing section 32a applies various image processing. That is, the image processing section 32a receives the image data outputted from the image pickup section (objective optical system 40, image pickup device 34) and executes image processing for generating image data for display (display signal).

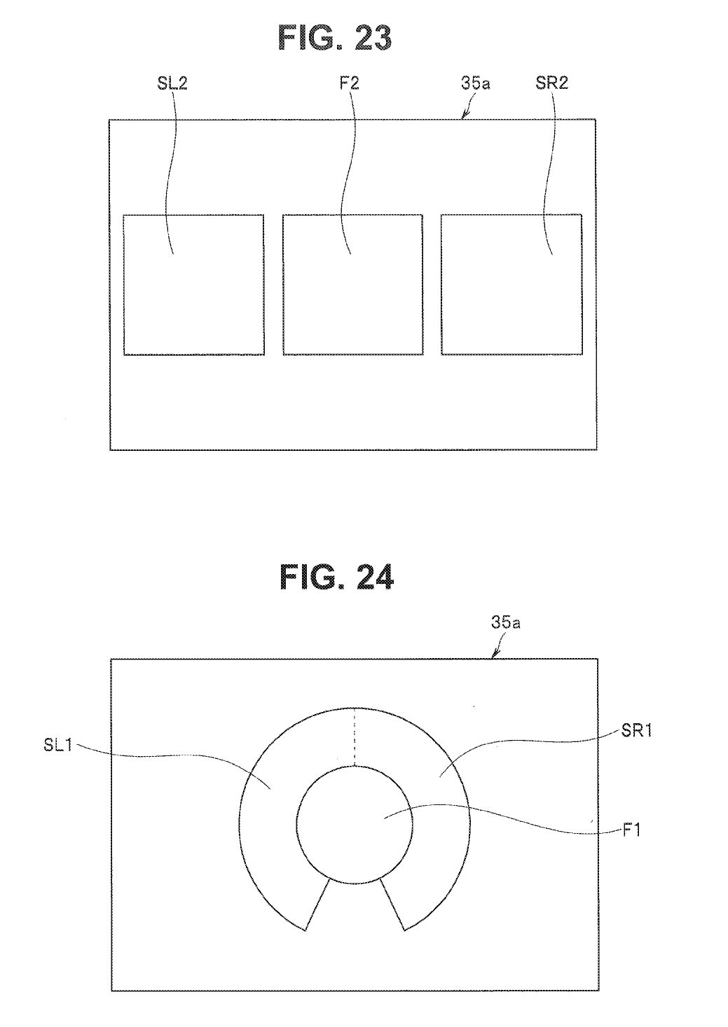

The endoscopic image for display generated in this way is transmitted to the display apparatus 35. In this case, the image processing section 32a displays, on the display screen 35a of the display apparatus 35, the endoscopic image in the second display mode (second mode) for display in the form of arranging the lateral field of view images (second field of view images) side by side with the forward image (first field of view image, F2) in the state in which the parts (SL2, SR2) of the lateral field of view images (second field of view images) including both sides of the forward image (first field of view image) are separated as shown in FIG. 23.

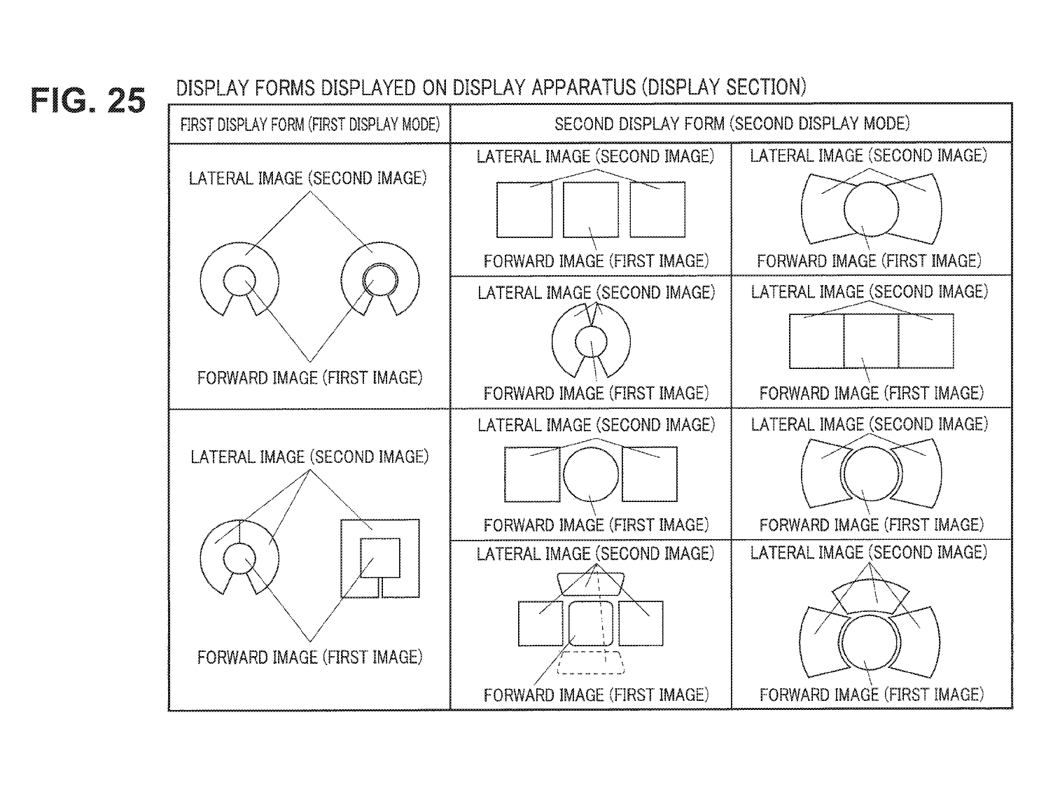

On the other hand, based on an instruction from the outside for example, the image processing section 32a makes a switch to the first display mode (first mode) for display in the form of arranging the lateral images (second field of view images) around the forward image (first field of view image, F1) in the state in which the parts (SL1, SR1) of the lateral images (second field of view images) including both sides of the forward image (first field of view image) are continuously connected as shown in FIG. 24.

That is, the image processing section 32a executes a mode switch control process of selectively switching between the second display mode (second mode) and the first display mode (first mode) and transmitting an image signal for display according to each display mode to the display apparatus 35 (display section). The mode switch control process by the image processing section 32a is appropriately executed at a predetermined timing based on, for example, an instruction from the outside.

The state in which the parts of the lateral images including both sides of the forward image are continuously connected denotes that parts of the lateral images are integrated. The state includes a state in which two lateral images are in contact with each other, a state in which two lateral images are integrated, and a state in which boundary processing is applied to the boundary of the two integrated or contacting lateral images. Examples of the boundary processing include image processing of making the border of the two lateral images inconspicuous and image processing of superimposing a thin boundary line on the border of the two lateral images.

The switch timing can also be changed in various ways as desired by the user in the second embodiment.

In the second embodiment, when the display is switched and shifted from the second display form (second display mode) to the first display form (first display mode), a correction process for the image in a designated region in the endoscopic image, such as a distortion correction process of the image displayed in a substantially circular shape and a changing process of the display shape (deformation process of deforming the rectangular image into the circular image and the like), may be executed.

As described, according to the second embodiment, the endoscope system 101 is configured to switch the display form of the endoscopic image displayed by using the display apparatus 35 between the display mode of arranging the lateral image side by side with the forward image in the state in which the parts of the lateral images including both sides of the forward image are separated and the display mode of arranging the lateral images around the forward image in the state in which the parts in the lateral images including both sides of the forward image are continuously connected. The endoscope system 101 switches the display form at a timing desired by the user or automatically switches the display form according to the operation form.

According to the configuration, when the endoscope system 101 is in use, the user can select an appropriate display form at a desired time. Therefore, an endoscope system that can be more easily used can be realized.

Note that it is obvious that the present invention is not limited to each of the embodiments described above, and various changes and applications can be made without departing from the scope of the invention. Furthermore, each of the embodiments includes inventions of various phases, and various inventions can be extracted based on appropriate combinations of a plurality of disclosed constituent conditions. For example, when the problem to be solved by the invention can be solved, and the advantageous effects can be obtained even if some of the constituent conditions illustrated in each of the embodiments are deleted, the configuration after the deletion of the constituent conditions can be extracted as an invention.

For example, various display forms can be considered as the display forms displayed on the display apparatus 35 (display section) as shown in FIG. 25, regarding the first display form (first display mode) of arranging the lateral images around the forward image in the state in which the parts of the lateral images including both sides of the forward image are continuously connected and the second display form (second display mode) of arranging the lateral images side by side with the forward image in the state in which the parts of the lateral images including both sides of the forward image are separated.

As for each of the display forms in the display modes switched respectively, an appropriate display form desired by the user can be selected from various display forms shown in FIG. 25.

### INDUSTRIAL APPLICABILITY

The present invention can be applied not only to an endoscope system of the medical field, but also to an endoscope system of the industrial field.

What is claimed is:

1. An endoscope system comprising:
an insertion portion inserted into a subject;
a support portion protruding forward from a distal end surface of the insertion portion;
a first subject image acquisition portion provided on the insertion portion and configured to acquire a first subject image from a forward region including a forward direction of the insertion portion;
a second subject image acquisition portion provided on the insertion portion and configured to acquire a second subject image from a lateral region including a radial direction of the insertion portion, the lateral region being at least partially different from the forward region;
a third subject image acquisition portion provided on the insertion portion and configured to acquire a third subject image from a third region that is a region in the forward region shielded by the support portion;
an image generation section configured to generate a forward image based on the first subject image, lateral images based on the second subject image, and a third image based on the third subject image; and
an image processing section configured to execute a process of arranging the lateral images around the forward image and arranging the third image in a non-display region of the lateral images generated by being shielded by the support portion, wherein
the image processing section can switch between a first mode of arranging the lateral images around the forward image in a state in which parts of the lateral images including both sides of the forward image are continuously connected and a second mode of arranging the lateral images side by side with the forward image in a state in which the parts including both sides of the forward image are separated, and
the image processing section executes a process of arranging the third image in the non-display region in a state in which the third image is continuously connected to the forward image in the first mode and a process of arranging the lateral images side by side with both sides of the forward image and arranging the third image adjacent to a region different from both sides of the forward image in the second mode.

2. The endoscope system according to claim 1, further comprising
a recording section configured to select a form of at least one of the first mode and the second mode to save an image signal including the forward image and the lateral images.

3. The endoscope system according to claim 1, wherein
the image processing section executes a process of hiding part of the forward image and the lateral images and converting the images such that the forward image, the lateral images, and the third image become rectangular in the second mode.

4. The endoscope system according to claim 1, wherein
the image processing section moves parallel or rotates and moves one of the forward image, the lateral images, and the third image to adjust a positional relationship between the forward image and the lateral images to display the forward image and the lateral images on a display section in the second mode.

5. The endoscope system according to claim 1, wherein
the image processing section expands or contracts one of the forward image, the lateral images, and the third image to adjust a size relationship among the forward image, the lateral images, and the third image to display the forward image, the lateral images, and the third image on a display section in the second mode.

6. The endoscope system according to claim 1, wherein
when the image processing section switches between the first mode and the second mode in a state in which part of a treatment instrument inserted into the insertion portion is displayed in one of the forward image and the lateral images, the image processing section executes a process of further displaying a trajectory of the treatment instrument not displayed in one of the first mode and the second mode.

7. The endoscope system according to claim 1, wherein
the image processing section removes distortion generated around one of the forward image and the lateral images to display the images on a display section.

8. The endoscope system according to claim 1, wherein
the image processing section switches between the first mode and the second mode based on one of a setting for gradually switching between the first mode and the second mode and a setting for instantaneously switching between the first mode and the second mode to display the images on a display section.

9. The endoscope system according to claim 1, wherein
the first subject image acquisition portion is arranged on a longitudinal direction distal end portion of the insertion portion, in a direction of the insertion of the insertion portion,
the second subject image acquisition portion is arranged to surround a circumferential direction of the insertion portion, and
the endoscope system further comprises an image pickup section arranged to photoelectrically convert the first subject image from the first subject image acquisition portion and the second subject image from the second subject image acquisition portion on a same surface, the image pickup section being electrically connected to the image processing section.

10. The endoscope system according to claim 1, wherein
the first subject image acquisition portion is arranged on a longitudinal direction distal end portion of the insertion portion, in a direction of the insertion of the insertion portion,
the second subject image acquisition portion is arranged on a side surface of the insertion portion, in a direction inclined relative to a longitudinal direction of the insertion portion, and
a first image pickup section configured to photoelectrically convert the first subject image from the first subject image acquisition portion and a second image pickup section configured to photoelectrically convert the second subject image from the second subject image acquisition portion are separately provided, and the first image pickup section and the second image pickup section are electrically connected to the image generation section.

11. The endoscope system according to claim 1, further comprising
a display section to which a signal based on the images from the image processing section is inputted, the display section being configured to display, based on one of the first mode and the second mode, endoscopic images respectively based on the forward image, the lateral images, and the third image.

12. The endoscope system according to claim 11, wherein
in the first mode,
the forward image is displayed in a substantially circular shape on the display section, and
the lateral images and the third image are displayed in a substantially annular shape surrounding the forward image on the display section.
